(19) United States
(12) Patent Application Publication
(10) Pub. No.: US 2024/0255508 A1
(43) Pub. Date: Aug. 1, 2024

(54) BIOMARKERS FOR RESPIRATORY DISEASE AND METHODS USING THE SAME

(71) Applicant: NATIONAL UNIVERSITY OF SINGAPORE, Singapore (SG)

(72) Inventors: Xinke LI, Singapore (SG); Yeow Meng CHEE, Singapore (SG); Ngeow Khing CHIA, Singapore (SG)

(21) Appl. No.: 18/565,509

(22) PCT Filed: May 31, 2022
(86) PCT No.: PCT/SG2022/050373

§ 371 Date: Nov. 29, 2023

(30) Foreign Application Priority Data
May 31, 2021 (SG) 10202105781X
Jul. 8, 2021 (SG) 10202107502P

(51) Int. Cl. G01N 33/569; G01N 33/53

(52) U.S. Cl. CPC G01N 33/56983; G01N 33/5308; G01N 2333/165

(57) ABSTRACT

The present application relates to methods of detecting the presence of a respiratory disease in a subject. The method comprising the step of detecting or measuring a concentration of two or more biomarkers selected from the group consisting of Water, Methanol, 1,3-Butadiene, Acetone, Isoprene, Isobutyronitrile, 3-Methylpyridine, Pentanoic Acid, m-Cresol or p-Cresol, N,N-Dimethylaniline, and n-Decane in a sample obtained from the subject. Other biomarkers may include Hydrogen Peroxide, Trimethylamine, Methacrolein, Butanal, Propanoic Acid, Butanol, Carbon Disulfide, Aniline, Furfural, Cycloheptene, 1-Octene, Benzoic Acid, 6-Methyl-5-Hepten-2-one, 1-Decene, Dodecane, β-Damascenone, Sesquiterpene, and Tetradecanoic Acid. The methods include applying the concentrations to a decision tree or a logistic regression model.

### CROSS REFERENCE TO RELATED APPLICATIONS

This application claims the benefit of Singapore Applications Nos. 10202105781X and 10202107502P filed on 31 May 2021 and 8 Jul. 2021, respectively, the disclosure of which is hereby incorporated in its entirety by reference herein.

### TECHNICAL FIELD

The present disclosure relates to the field of respiratory disease. In particular, the disclosure provides biomarkers for respiratory disease and methods based on the same biomarkers.

### BACKGROUND

Respiratory disease may be caused by respiratory infections. Respiratory infections are a type of infections caused by pathogens, including viruses and bacteria, that affect the respiratory tract, including the nose, windpipe, and lungs. Examples of respiratory viruses include adenovirus, enterovirus, human coronavirus, rhinovirus, and influenza. Examples of respiratory bacteria include Streptococcus pneumoniae, Mycoplasma pneumoniae, Haemophilus influenzae, and Chlamydophila pneumoniae. Although respiratory tract infections are usually self-limiting and confined to the upper respiratory tract, respiratory tract infections may in more severe cases affect the lower respiratory tract leading to symptoms like pneumonia and bronchiolitis.

In general, many respiratory pathogens cause seasonal respiratory infections in temperate climates, with a large increase in infections during colder months. However, in the more modern era, there has been multiple cases of epidemics and pandemics due to novel respiratory viruses emerging with increasing frequency. Examples include the severe acute respiratory syndrome coronavirus (SARS-CoV) causing the SARS epidemic, the Middle East respiratory syndrome-related coronavirus causing the MERS outbreak (MERS-CoV), the influenza A H1N1 causing the 2009 swine flu pandemic, and the severe acute respiratory syndrome coronavirus 2 (SARS-CoV-2) causing the current coronavirus disease 2019 (COVID-19) pandemic.

Current testing options for respiratory viruses include pathogen culture, rapid antigen detection tests (RADTs), direct fluorescent antibody (DFA), and nucleic acid-amplification (NAAT) tests. However, such laboratory tests are relatively costly and have a long turnaround time and thus are not sufficiently efficient in a time of an epidemic or pandemic which requires rapid testing of a high volume of individuals. Furthermore, many of these tests require the collection of samples through a nasal or throat swab, which is uncomfortable for the subject being tested. For example, swab-based NAATs detect, in a symptomatic patient, viral shedding in the form of viral specific sequences of RNA. Although such tests are highly specific, they typically require a minimum of 3-4 hours (including specimen transport, extraction, NAAT itself) for a confirmatory result which makes such tests unsuitable for mass screening events involving a large number of persons. Testing of individuals is particularly important in a time of an epidemic and pandemic so that suspected infected individual may be quickly identified and isolated to prevent the spread of infection. It is also important as infected individuals may be asymptomatic and still able to pass the respiratory virus to others. There is therefore a need for a method of testing for respiratory disease, particularly respiratory disease caused by respiratory viruses, that is inexpensive, rapid, and sufficiently accurate to identify infected individuals.

Volatile organic compounds (VOCs) in exhaled breath are biomarkers that have gained interest as precise measurement of exhaled VOCs at extremely low concentrations was made possible recently by advances in analytical instruments and breath sampling devices, such that measurement may be carried out in real-time with results obtained within a minute. Volatile organic compounds (VOCs) are produced by various metabolic processes, and they reflect the physiological status of the human body. Certain VOCs in exhaled breath have already been shown to be associated with disease conditions such as lung cancer, tuberculosis, as well as influenza. Furthermore, there is strong evidence that suggests that viral infections result in in increased oxidative stress, which in turn, will result in increased levels of VOC biomarkers such as alkanes and aldehydes in the exhaled breath. Oxidative stress is the overall balance between formation and scavenging of reactive oxygen species (ROS) and free radicals. Previous research on biochemical pathways of VOCs in human breath suggest that increased level of ROS leads to increased levels of peroxidation of fatty acids, resulting in elevated level of alkanes in exhaled breath. Therefore, volatile biomarkers in exhaled breath would allow rapid non-invasive screening of respiratory infections which would allow mass screening of individuals for possible respiratory infections and identify individuals to carry out further confirmatory tests on.

### SUMMARY

Methods of detecting the presence of a respiratory disease in a subject is provided. According to some embodiments, the method comprises the step of detecting or measuring a concentration of Acetone and 1-Propanol in a sample obtained from the subject. The panel of biomarkers optionally comprises Acetone at about 1.4 times increase and 1-Propanol at about 1.2 times increase in comparison with a reference. The panel of biomarkers optionally comprises Acetone having a m/z value being 59.05±1 and 1-Propanol having a m/z value being 61.10±1. Optionally, the method may further comprise the step of detecting or measuring a concentration of: Nonanal, 3-(Ethylthio)propanal, Butyraldehyde, Acetaldehyde, and 3-Hydroxybutyric Acid.

According to some embodiments, the method comprises the step of detecting or measuring a panel of biomarkers comprising Butanol and Isoprene in a sample obtained from the subject. The panel of biomarkers optionally comprises Butanol at about 3 times increase and Isoprene at about 1.8 times increase in comparison with a reference. The panel of biomarkers optionally comprises Butanol having a m/z value being 75.12±1 and Isoprene having a m/z value being 69.07±1. Optionally, the panel of biomarkers further comprising Acetone, Cycloheptene, and Sesquiterpene.

According to some embodiments, the method comprising the step of detecting or measuring a panel of biomarkers comprising 1,3-Butadiene and Acetone in a sample obtained from the subject. The panel of biomarkers optionally comprises 1,3-Butadiene at about 1.5 times increase and Acetone at about 1.7 times increase in comparison with a reference. The panel of biomarkers optionally comprises 1,3-Butadiene having a m/z value being 55.05±1 and Acetone having a m/z value being 59.05±1. Optionally, the panel of biomarkers further comprising Isoprene, Methacrolein, Butanal, Propanoic Acid, Aniline, Furfural, 1-Octene, 6-Methyl-5-Hepten-2-one, 1-Decene, Dodecane, β-Damascenone, Sesquiterpene, and Tetradecanoic Acid.

According to some embodiments, the method comprising the step of detecting or measuring a panel of biomarkers comprising Hydrogen Peroxide and Water in a sample obtained from the subject. Optionally, the panel of biomarkers further comprising 1,3-Butadiene, Acetone, Trimethylamine, Isoprene, Carbon Disulfide, 3-Methylpyridine, Benzoic Acid, and Tetradecanoic Acid.

Optionally, the said biomarkers are measured by proton transfer reaction time-of-flight mass spectrometry. Optionally, the respiratory disease is a coronavirus infection. Optionally, the respiratory disease is COVID-19. Optionally, an increased or decreased level of each of the biomarker as compared to a reference indicates the presence of the respiratory disease in the subject. Optionally, the reference is obtained from a subject or group of subjects without the respiratory disease. Optionally, the method further comprises treating the respiratory disease by administering a pharmaceutical composition to the subject.

There is further provided a method of detecting the presence of a respiratory disease in a subject, the method comprising the steps of collecting a breath sample from a subject; detecting in said breath sample two or more biomarkers and measuring a concentration of the two or more biomarkers selected from the group consisting of Water, Methanol, 1,3-Butadiene, Acetone, Isoprene, Isobutyronitrile, 3-Methylpyridine, Pentanoic Acid, m-Cresol or p-Cresol, N,N-Dimethylaniline, and n-Decane; and determining the presence of a respiratory disease.

There is further provided a method of detecting the presence of a respiratory disease in a subject, the method comprising the step of detecting or measuring a concentration of n-Decane and Isobutyronitrile. Optionally, the method may further comprise the step of detecting or measuring a concentration of Isoprene.

### BRIEF DESCRIPTION OF THE DRAWINGS

In order for the present disclosure, to be better understood and for its practical applications to be appreciated, the following Figures are provided and referenced hereafter. It should be noted that the Figures are given as examples only and in no way limit the scope of the invention.

FIG. 1 is a schematic illustration of a decision tree generated using data from samples collected through direct exhalation, in accordance with embodiments of the present disclosure.

FIG. 2 is a schematic illustration of a respiratory cycle segmented into phases including background and exhaling, in accordance with embodiments of the present disclosure.

FIG. 3 is a schematic illustration of the selected features with significant importance and their molecular weight (MW) obtained from a machine learning model, in accordance with embodiments of the present disclosure.

Identical or duplicate or equivalent or similar structures, elements, or parts that appear in one or more drawings are generally labelled with the same reference numeral, optionally with an additional letter or letters to distinguish between similar entities or variants of entities and may not be repeatedly labelled and/or described. References to previously presented elements are implied without necessarily further citing the drawing or description in which they appear.

### DETAILED DESCRIPTION

The present disclosure provides biomarkers for respiratory disease and methods based on the same biomarkers. The term “respiratory disease” used herein refers to or describes the presence of any disease infecting the respiratory tract of a subject, including the nose, windpipe, and lungs. The respiratory disease may be a respiratory pathogen infection, including respiratory viral infection and respiratory bacterial infection. An example of respiratory disease is respiratory pathogen infection caused by a respiratory virus or a respiratory bacterium. An example of a respiratory virus is SARS-CoV-2 and an example of a respiratory disease is COVID-19. When the respiratory disease is COVID-19, the COVID-19 may be any variants of COVID-19 including the following variants: alpha, beta, gamma, delta, omicron including their subvariants thereof.

As used herein, the term “biomarker” means a compound, including a metabolite, that is differentially present (i.e., increased or decreased) in a biological sample obtained from a subject or a group of subjects having a first phenotype (e.g., having a disease) as compared to a biological sample obtained from a subject or group of subjects having a second phenotype (e.g., not having the disease). The term “panel of biomarkers” may refer to two or more biomarkers. The biomarkers may be VOCs. Non-limiting examples of the biomarkers include Water, Methanol, 1,3-Butadiene, Acetone, Isoprene, Isobutyronitrile, 3-Methylpyridine, Pentanoic Acid, m-Cresol or p-Cresol, N,N-Dimethylaniline, n-Decane, Butanol, Cycloheptene, Sesquiterpene, 1,3-Butadiene, Methacrolein, Butanal, Propanoic Acid, Aniline, Furfural, 1-Octene, 6-Methyl-5-Hepten-2-one, 1-Decene, Dodecane, β-Damascenone, Tetradecanoic Acid, Hydrogen Peroxide, Trimethylamine, Carbon Disulfide, 3-Methylpyridine, Benzoic Acid, 1-Propanol, Nonanal, 3-(Ethylthio)propanal, Butyraldehyde, Acetaldehyde, and 3-Hydroxybutyric Acid.

As used herein, the term “increase” or “increased’ with reference to a biomarker refers to a statistically significant and measurable increase in the biomarker as compared to a control or reference. The increase is preferably an increase of at least about 10%, or an increase of at least about 20%, or an increase of at least about 30%, or an increase of at least about 40%, or an increase of at least about 50%.

In some embodiments, an increased level of each of the biomarker as compared to a control indicates the presence of a respiratory disease in the subject. The increase in level may be an increase of 1.1 times, 1.2 times, 1,3 times, 1.4 times, 1.5 times, 1.6 times, 1.7 times, 1.8 times, 1.9 times, 2 times, 3 times, 4 times, 5 times, 6 times, 7 times, 8 times, 9 times, 10 times, 11 times 12 times, 13 times, 14 times, 15 times, 16 times, 17 times, 18 times, 19 times, 20 times, 21 times, 22 times, 23 fold, 24 times, 25 times, 26 times, 27 times, 28 times, 29 times, 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, 60 times, 61 times, 62 times, 63 times, 64 times, 65 times, 66 times, 67 times, 68 times, 69 times, 70 times, 71 times, 72 times, 73 times, 74 times, 75 times, 76 times, 77 times, 78 times, 79 times, 80 times, 81 times, 82 times, 83 times, 84 times, 85 times, 86 times, 87 times, 88 times, 89 times, 90 times, 91 times, 92 times, 93 times, 94 times, 95 times, 96 times, 97 times, 98 times, 99 times or 100 times or anywhere in between as compared to a control.

As used herein, the term “decrease” or “decreased’ with reference to a biomarker refers to a statistically significant and measurable decrease in the biomarker as compared to a control or reference. The decrease is preferably a decrease of at least about 10%, or a decrease of at least about 20%, or a decrease of at least about 30%, or a decrease of at least about 40%, or a decrease of at least about 50%.

In some embodiments, a decreased level of each of the biomarker as compared to a control indicates the presence of a respiratory disease in the subject. The decrease in level may refer to a biomarker having 0.9 times or less, 0.8 times or less, 0.75 times or less, 0.7 times or less, 0.6 times or less, 0.5 times or less, 0.4 times or less, 0.3 times or less, 0.2 times or less, 0.16 times or less, 0.1 times or less or anywhere in between as compared to the level of a control.

The control or reference may be a sample obtained from a subject who is healthy. The control may also be samples obtained from a group of subjects who are healthy. Each healthy subject may be one who does not have respiratory disease, whose breath data is used as a reference.

The term “subject” as used throughout the specification is to be understood to mean a human. The “subject” may include a person, a patient, a participant, or individual, and may be of any age, race or gender.

As used herein, the term “sample” includes tissues, cells, body fluids and isolates thereof etc., isolated from a subject, as well as tissues, cells, and fluids etc. present within a subject (i.e., the sample is in vivo). Non-limiting examples of samples include whole blood, blood fluids (e.g., serum and plasm), lymph and cystic fluids, sputum, stool, tears, mucus, hair, skin, breath (e.g., exhaled breath), ascitic fluid, cystic fluid, urine, nipple exudates, nipple aspirates, sections of tissues such as biopsy and autopsy samples, frozen sections taken for histologic purposes, archival samples, explants and primary and/or transformed cell cultures derived from patient tissues etc.

The term “Limit of Detection or LOD” or “sensitivity” as used herein refers to the lowest concentration of the biomarkers that can be detected by an analytical instrument. The lowest concentration is statistically distinguishing from background or noise. These terms may therefore be used interchangeably throughout the disclosure. The LOD or sensitivity may be specific to the analytical instrument used and may be limited to their calibrated LOD for each analyte. Non-limiting example of the analyte in the present disclosure includes VOCs.

In one embodiment of the present disclosure, there is a method of detecting a presence of respiratory disease by measuring a panel of biomarkers comprising Water, Methanol, 1,3-Butadiene, Acetone, Isoprene, Isobutyronitrile, 3-Methylpyridine, Pentanoic Acid, m-Cresol or p-Cresol, N,N-Dimethylaniline, and n-Decane. Water may be water, moisture, vapour, water droplets, or any other form of water. The panel of biomarkers may comprise any number of biomarkers from the group such as 2, 3, 4, 5, 6, 7, 8, 9, 10 or 11 biomarkers.

In some embodiments of the present disclosure, the panel of biomarkers may comprise n-Decane and Isobutyronitrile. In some embodiments of the present disclosure, the panel of biomarkers may comprise n-Decane at about 0.7 times decrease and Isobutyronitrile at about 1.3 times increase in comparison with a reference. In some embodiments of the present disclosure, the panel of biomarkers may comprise n-Decane having m/z value being 143.18±1 and Isobutyronitrile having m/z value being 70.07±1.

In some embodiments of the present disclosure, the panel of biomarkers may comprise n-Decane, Isobutyronitrile and Isoprene. In some embodiments of the present disclosure, the panel of biomarkers may comprise n-Decane at about 0.7 times decrease, Isobutyronitrile at about 1.3 times increase, and Isoprene at about 1.1 times increase in comparison with a reference. In some embodiments of the present disclosure, the panel of biomarkers may comprise n-Decane having m/z value being 143.18±1, Isobutyronitrile having m/z value being 70.07±1 and Isoprene having m/z value being 69.08±1.

In some embodiments, the panel of biomarkers may be selected from the group consisting of Water, Methanol, 1,3-Butadiene, Acetone, Isoprene, Isobutyronitrile, 3-Methylpyridine, Pentanoic Acid, m-Cresol or p-Cresol, N,N-Dimethylaniline, and n-Decane. The panel of biomarkers may be detected from a sample obtained from a subject. The panel of biomarkers may be detected from a sample of exhaled breath obtained from a subject. An increase or decrease of the level of each of the biomarker as compared to a control may indicate the presence of a respiratory disease in the subject. In some embodiments, the method may be a qualitative method for detecting the panel of biomarkers. In some embodiments, the method disclosed herein may be a quantitative method for detecting the panel of biomarkers and measuring the concentration of the biomarkers. In some embodiments, the method may comprise detecting m/z of the biomarkers comprising m/z values being 38.04±1, 51.04±1, 55.05±1, 59.05±1, 69.08±1, 70.07±1, 94.07±1, 103.08±1, 109.07±1, 121.96±1, and 143.18±1. In some embodiments, the m/z values being signature m/z values that correspond to Water (Cluster), Methanol (Water Cluster), 1,3-Butadiene, Acetone, Isoprene, Isobutyronitrile, 3-Methylpyridine, Pentanoic Acid, m-Cresol or p-Cresol, N,N-Dimethylaniline, and n-Decane, respectively. In some embodiments, the panel of biomarkers may further comprise the following biomarkers with increase or decrease level of concentration: Water (Cluster) at 1.3 times increase, Methanol (Water Cluster) at 2.6 times increase, 1,3-Butadiene at 2.5 times increase, Acetone at 1.5 times increase, 3-Methylpyridine at 1.7 times increase, Pentanoic Acid at 0.3 times decrease, m-Cresol or p-Cresol at 2.5 times increase, and N,N-Dimethylaniline at 1.3 times increase. In some embodiments, the increase in the level of each of the biomarkers in comparison with a control or reference may be as follows: Water (Cluster) from about 1.3 times to about 100 times, Methanol (Water Cluster) from 2.6 times to 100 times, 1,3-Butadiene from 2.5 times to 100 times, Acetone from 1.5 times to 100 times, Isoprene from 1.1 times to 100 times, Isobutyronitrile from 1.3 times to 100 times, 3-Methylpyridine from 1.7 times to 100 times, m-Cresol or p-Cresol from 2.5 times to 100 times and N,N-Dimethylaniline from 1.3 times to 100 times. It is understood that any range described in paragraphs [0021] and [0022] of the present application may be used to define narrower ranges. In some embodiments, the decrease in the level of each of the biomarkers in comparison with a control or reference may be as follows: n-Decane from 0.7 times to 0.1 times and Pentanoic Acid from 0.3 times to 0.1 times. It is understood that any range described in paragraphs [0023] and [0024] of the present application may be used to define narrower ranges.

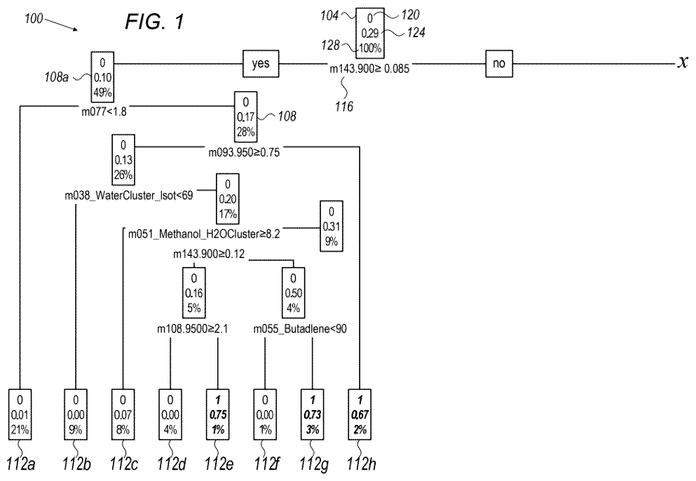

In some embodiments of the present disclosure, the method may further comprise applying the levels of biomarkers measured to a decision tree generated using data obtained from subjects without the respiratory disease and subjects with the respiratory disease. The decision tree may be generated using suitable algorithms, including ID3 (Iterative Dichotomiser 3), C4.5, CART (Classification and Regression Tree), CHAID (Chi-square automatic interaction detection) and MARS (multivariate adaptive regression splines). Other suitable algorithms may also be used. An example of a decision tree generated using data is that depicted in FIG. 1. FIG. 1 is a schematic illustration of a decision tree 100 generated using data from samples collected through direct exhalation, in accordance with embodiments of the present disclosure. The decision tree 100 comprises a root node 104, intermediate nodes 108, and twenty-six terminal nodes 112 corresponding to a classification of either presence or absence of respiratory disease. Root node 104 and intermediate nodes 108 comprise biomarker-based decision rules based on a concentration of a biomarker. Terminal nodes 112c, 112g, 112h, 112k, 112l, 112o, 112p, 112t, 112u, 112v, 112x, and 112z correspond to a classification of positive or presence of respiratory disease. Terminal nodes 112a, 112b, 112c, 112d, 112f, 112i, 112j, 112m, 112n, 112q, 112r, 112s, 112w, and 112y correspond to a classification of negative or absence of respiratory disease. The decision tree 100 includes all eleven biomarkers, although not all biomarkers may be required to reach a terminal node 112. In one example, depending on the concentration of each biomarker, biomarkers n-Decane and 3-Methylpyridine may be sufficient to reach terminal node 112q with a classification of absence of respiratory disease. In another example, biomarkers n-Decane, Isobutyronitrile, and 3-Methylpyridine may be sufficient to reach terminal node 112h with a classification of presence of respiratory disease.

The rules which make up decision tree 100 are:
(1) if n-Decane ≥0.085 and Isobutyronitrile <1.8, the decision is terminal node 112a with a classification of negative;
(2) if n-Decane ≥0.085, Isobutyronitrile ≥1.8, 3-Methylpyridine ≥0.75, and water <69, the decision is terminal node 112b with a classification of negative;
(3) if n-Decane ≥0.085, Isobutyronitrile ≥1.8, 3-Methylpyridine ≥0.75, water ≥69 and Methanol (water cluster)>8.2, the decision is terminal node 112c with a classification of negative;
(4) if n-Decane ≥0.085, Isobutyronitrile ≥1.8, 3-Methylpyridine ≥0.75, water ≥69, Methanol (water cluster)<8.2, n-Decane >0.12, and m-cresol or p-cresol ≥2.1, the decision is terminal node 112d with a classification of negative;
(5) if n-Decane ≥0.085, Isobutyronitrile ≥1.8, 3-Methylpyridine ≥0.75, water ≥69, Methanol (water cluster)<8.2, n-Decane ≥0.12, and m-cresol or p-cresol <2.1, the decision is terminal node 112e with a classification of positive;
(6) if n-Decane ≥0.085, Isobutyronitrile ≥1.8, 3-Methylpyridine ≥0.75, water ≥69, Methanol (water cluster)<8.2, n-Decane <0.12, and 1,3-Butadiene <90, the decision is terminal node 112f with a classification of negative;
(7) if n-Decane ≥0.085, Isobutyronitrile ≥1.8, 3-Methylpyridine ≥0.75, water ≥69, Methanol (water cluster)<8.2, n-Decane <0.12, and 1,3-Butadiene ≥90, the decision is terminal node 112g with a classification of positive;
(8) if n-Decane ≥0.085, Isobutyronitrile ≥1.8, and 3-Methylpyridine <0.75, the decision is terminal node 112h with a classification of positive;
(9) if n-Decane <0.085, 3-Methylpyridine <0.34, Isoprene <122, Methanol (water cluster)<8.5, N,N-Dimethylaniline <0.15, the decision is terminal node 112i with a classification of negative;
(10) if n-Decane <0.085, 3-Methylpyridine <0.34, Isoprene <122, Methanol (water cluster)<8.5, N,N-Dimethylaniline ≥0.15, Pentanoic Acid <0.13, the decision is terminal node 112j with a classification of negative;
(11) if n-Decane <0.085, 3-Methylpyridine <0.34, Isoprene <122, Methanol (water cluster)<8.5, N,N-Dimethylaniline ≥0.15, Pentanoic Acid ≥0.13, the decision is terminal node 112k with a classification of positive;
(12) if n-Decane <0.085, 3-Methylpyridine <0.34, Isoprene <122, and Methanol (water cluster) ≥8.5, the decision is terminal node 112l with a classification of positive;
(13) if n-Decane <0.085, 3-Methylpyridine <0.34, Isoprene ≥122, water <22, and Isobutyronitrile ≥1.2, the decision is terminal node 112m with a classification of negative;
(14) if n-Decane <0.085, 3-Methylpyridine <0.34, Isoprene ≥122, water <22, Isobutyronitrile <1.2, and Acetone ≥811, the decision is terminal node 112n with a classification of negative;
(15) if n-Decane <0.085, 3-Methylpyridine <0.34, Isoprene ≥122, water <22, Isobutyronitrile <1.2, and Acetone <811, the decision is terminal node 112o with a classification of positive;
(16) if n-Decane <0.085, 3-Methylpyridine <0.34, Isoprene ≥122, and water ≥22, the decision is terminal node 112p with a classification of positive;
(17) if n-Decane <0.085, 3-Methylpyridine >0.34, and n-Decane <0.057, the decision is terminal node 112q with a classification of negative;
(18) if n-Decane <0.085, 3-Methylpyridine ≥0.34, n-Decane ≥0.057, Methanol (water cluster)<3.7, water ≥20, N,N-Dimethylaniline <0.17, N,N-Dimethylaniline ≥0.13, and N,N-Dimethylaniline <0.15, the decision is terminal node 112r with a classification of negative;
(19) if n-Decane <0.085, 3-Methylpyridine ≥0.34, n-Decane ≥0.057, Methanol (water cluster)<3.7, water ≥20, N,N-Dimethylaniline <0.17, N,N-Dimethylaniline >0.13, N,N-Dimethylaniline >0.15, and Methanol (water cluster)<2.9, the decision is terminal node 112s with a classification of negative;
(20) if n-Decane <0.085, 3-Methylpyridine ≥0.34, n-Decane ≥0.057, Methanol (water cluster)<3.7, water ≥20, N,N-Dimethylaniline <0.17, N,N-Dimethylaniline ≥0.13, N,N-Dimethylaniline >0.15, and Methanol (water cluster) ≥2.9, the decision is terminal node 112t with a classification of positive;
(21) if n-Decane <0.085, 3-Methylpyridine ≥0.34, n-Decane ≥0.057, Methanol (water cluster)<3.7, water >20, N,N-Dimethylaniline <0.17, and N,N-Dimethylaniline <0.13, the decision is terminal node 112u with a classification of positive;
(22) if n-Decane <0.085, 3-Methylpyridine ≥0.34, n-Decane ≥0.057, Methanol (water cluster)<3.7, water ≥20, and N,N-Dimethylaniline ≥0.17, the decision is terminal node 112v with a classification of positive;
(23) if n-Decane <0.085, 3-Methylpyridine ≥0.34, n-Decane ≥0.057, Methanol (water cluster)<3.7, water <20, and 1,3-Butadiene <31, the decision is terminal node 112w with a classification of negative;
(24) if n-Decane <0.085, 3-Methylpyridine ≥0.34, n-Decane ≥0.057, Methanol (water cluster)<3.7, water <20, and 1,3-Butadiene ≥31, the decision is terminal node 112x with a classification of positive;
(25) if n-Decane <0.085, 3-Methylpyridine ≥0.34, n-Decane ≥0.057, Methanol (water cluster) ≥3.7, and Isobutyronitrile <1.3, the decision is terminal node 112y with a classification of negative; and
(26) if n-Decane <0.085, 3-Methylpyridine >0.34, n-Decane ≥0.057, Methanol (water cluster) ≥3.7, and Isobutyronitrile ≥1.3, the decision is terminal node 112z with a classification of positive, wherein the values expressed are concentrations of the biomarkers in parts per billion (ppb).

In another embodiment of the present disclosure, the method may comprise detecting or measuring a panel of biomarkers comprising Butanol, Isoprene, Acetone, Cycloheptene, and Sesquiterpene. The panel of biomarkers may comprise any number of biomarkers from the group. In some embodiments, the panel of biomarkers may comprise Butanol, Isoprene, Acetone, Cycloheptene, and Sesquiterpene.

In another embodiment of the present disclosure, the method may comprise detecting or measuring a panel of biomarkers comprising Butanol and Isoprene. In some embodiments of the present disclosure, the panel of biomarkers may comprise Butanol at about 3 times increase and Isoprene at about 1.8 times increase in comparison with a reference. In some embodiments of the present disclosure, the panel of biomarkers may comprise Butanol having m/z value being 75.12±1 and Isoprene having m/z value being 69.07±1.

In some embodiments, the panel of biomarkers may further comprise Acetone, Cycloheptene, and Sesquiterpene. In some embodiments, Acetone may be at about 1.7 times increase, Cycloheptene may be at about 1.5 times increase and Sesquiterpene may be at about 1.5 times increase in comparison with a reference. In some embodiments, the panel of biomarkers may further comprise Acetone having m/z value being 59.05±1, Cycloheptene having m/z value being 97.10±1, and Sesquiterpene having m/z value being 205.20±1.

In some embodiments, the panel of biomarkers may comprise Butanol, Isoprene, and Acetone. In some embodiments, the panel of biomarkers may comprise Butanol, Isoprene, and

Cycloheptene. In some embodiments, the panel of biomarkers may comprise Butanol, Isoprene, Acetone and Cycloheptene. It is to be understood that other combinations of 3 or 4 biomarkers than the above may be used for the detecting the presence of respiratory disease.

The panel of biomarkers may be detected from a sample obtained from a subject. The panel of biomarkers may be detected from a sample of exhaled breath from a subject. An increase or decrease of the level of each of the biomarker as compared to a control may indicates the presence of a respiratory disease in the subject. In some embodiments of the present disclosure, the method may further comprise applying the levels of biomarkers measured to a decision tree generated using data obtained from subjects without the respiratory disease and subjects with the respiratory disease.

In some embodiments, the method may comprise detecting m/z of the biomarkers comprising m/z values being 75.12±1, 69.07±1, 59.05±1, 97.10±1, and 205.20±1. In some embodiments, the m/z values being signature m/z values that correspond to Butanol, Isoprene, Acetone, Cycloheptene, and Sesquiterpene, respectively. In some embodiments, the panel of biomarkers may further comprise the following biomarkers with increased level of concentration in comparison with a control or reference: Cycloheptene at 1.5 times increase and Sesquiterpene at 1.5 times increase. In some embodiments, the increase in the level of each of the biomarkers in comparison with a control or reference may be as follows: Butanol from about 2 or 3 times to about 100 times, Isoprene from 1.8 times to 100 times, Acetone from about 1.7 times to 100 times, Cycloheptene from about 1.5 times to 100 times, and Sesquiterpene from 1.5 times to 100 times. It is understood that any range described in paragraphs [0021] and [0022] of the present application may be used to define narrower ranges.

In some embodiments of the present disclosure, the method may further comprise applying the levels of biomarkers measured to a logistic regression model with equation (i) as follows:

$$y = \qquad ( i )$$

$$\frac{1}{\begin{matrix}1 + \\ {e}^{- ( - 2.03 + 1.69 * sesquiterpene + 1.189 * cycloheptene - 0.3 * butanol - 0.002 * isoprene - 0.003 * acetone )}\end{matrix}}$$

where a sample with a y-value higher than a defined cut-off value was classified as positive for respiratory disease.

In some embodiments, the defined cut-off value, or COP, is 0.2, such that any samples with a y-value higher than 0.2 was classified as positive for respiratory disease.

In another embodiment of the present disclosure, the method may comprise detecting or measuring a panel of biomarkers comprising 1,3-Butadiene, Acetone, Isoprene, Methacrolein, Butanal, Propanoic Acid, Aniline, Furfural, 1-Octene, 6-Methyl-5-Hepten-2-one, 1-Decene, Dodecane, β-Damascenone, Sesquiterpene, and Tetradecanoic Acid. The panel of biomarkers may comprise any number of biomarkers from the group above (having 15 biomarkers) for example, 2, 3, 4, 5, 6, 7, 8, 9, 10, 11, 12, 13, 14 or 15 biomarkers.

In some embodiments, the panel of biomarkers may comprise 1,3-Butadiene and Acetone. In some embodiments of the present disclosure, the panel of biomarkers may comprise 1,3-Butadiene at about 1.5 times increase and Acetone at about 1.7 times increase in comparison with a reference. In some embodiments of the present disclosure, the panel of biomarkers may comprise 1,3-Butadiene having m/z value being 55.05±1 and Acetone having m/z value being 59.05±1.

In some embodiments, the panel of biomarkers may comprise 1,3-Butadiene, Acetone and Isoprene. In some embodiments of the present disclosure, the panel of biomarkers may comprise 1,3-Butadiene at about 1.5 times increase, Acetone at about 1.7 times increase and Isoprene at about 1.8 times increase in comparison with a reference. In some embodiments of the present disclosure, the panel of biomarkers may comprise 1,3-Butadiene having m/z value being 55.05±1, Acetone having m/z value being 59.05±1 and Isoprene having m/z value being 69.08±1.

In some embodiments, the panel of biomarkers may comprise 1,3-Butadiene, Acetone, Isoprene, and Methacrolein. In some embodiments, the panel of biomarkers may comprise 1,3-Butadiene, Acetone, Isoprene, Methacrolein, and Butanal. In an exemplary embodiment, the panel of biomarkers may be selected from the group consisting of 1,3-Butadiene, Acetone, Isoprene, Methacrolein, Butanal, Propanoic Acid, Aniline, Furfural, 1-Octene, 6-Methyl-5-Hepten-2-one, 1-Decene, Dodecane, β-Damascenone, Sesquiterpene, and Tetradecanoic Acid.

In an exemplary embodiment, the panel of biomarkers may be selected from the group consisting of 1,3-Butadiene, Acetone, Isoprene, Methacrolein, Butanal, Propanoic Acid, Aniline, Furfural, 1-Octene, 6-Methyl-5-Hepten-2-one, 1-Decene, Dodecane, β-Damascenone, and Sesquiterpene. In another exemplary embodiment, the panel of biomarkers may be selected from the group consisting of 1,3-Butadiene, Acetone, Isoprene, Methacrolein, Butanal, Propanoic Acid, Aniline, Furfural, 1-Octene, 6-Methyl-5-Hepten-2-one, 1-Decene, Dodecane, and β-Damascenone. The panel of biomarkers may be detected from a sample obtained from a subject. The panel of biomarkers may be detected from a sample of exhaled breath from a subject. An increase or decrease of the level of each of the biomarker as compared to a control may indicates the presence of a respiratory disease in the subject. In some embodiments of the present disclosure, the method may further comprise applying the levels of biomarkers measured to a decision tree generated using data obtained from subjects without the respiratory disease and subjects with the respiratory disease. In some embodiments, the method may comprise detecting m/z of the biomarkers comprising m/z values being 55.05±1, 59.05±1, 69.08±1, 71.05±1, 73.06±1, 75.04±1, 94.07±1, 97.03±1, 113.13±1, 127.11±1, 141.16±1, 171.21±1, 191.14±1, 205.20±1, and 229.22±1. In some embodiments, the m/z values being signature m/z values that correspond to 1,3-Butadiene, Acetone, Isoprene, Methacrolein, Butanal, Propanoic Acid, Aniline, Furfural, 1-Octene, 6-Methyl-5-Hepten-2-one, 1-Decene, Dodecane, β-Damascenone, Sesquiterpene, and Tetradecanoic Acid, respectively. In some embodiments, the panel of biomarkers may further comprise the following biomarkers with increase or decrease level of concentration: Methacrolein (at 0.6 times decrease), Butanal (at 0.5 times decrease), Propanoic Acid (at 4 times increase), Aniline (at 1.8 times increase), Furfural (at 0.7 times decrease), 1-Octene (at 1.6 times increase), 6-Methyl-5-Hepten-2-one (at 0.16 times decrease), 1-Decene (at 8 times increase), Dodecane (at 5 times increase), β-Damascenone (at 17 times increase), Sesquiterpene (at 1.3 times increase), and Tetradecanoic Acid (at 1.8 times increase). In some embodiments, the increase in the level of each of the biomarkers in comparison with a control or reference may be as follows: 1,3-Butadiene from about 1.5 times to 100 times, Acetone from about 1.7 times to 100 times, Isoprene from about 1.8 times to about 100 times, Propanoic Acid from about 4 times to 100 times, Aniline from about 1.8 times to 100 times, 1-Octene from 1.6 times to 100 times, 1-Decene from about 8 times to 100 times, Dodecane from about 5 times to 100 times, β-Damascenone from about 17 times to 100 times, Sesquiterpene from about 1.3 times to about 100 times, and Tetradecanoic Acid from about 1.8 times to 100 times. It is to be understood that any range described in paragraphs [0021] and [0022] of the present application may be used to define narrower ranges. In some embodiments, the decrease in the level of each of the biomarkers in comparison with a control or reference may be as follows: Methacrolein from 0.6 times to 0.1 times, Butanal from 0.5 times to 0.1 times, Furfural from 0.7 times to 0.1 times, and 6-Methyl-5-Hepten-2-one from 0.16 times to 0.1 times. It is understood that any range described in paragraphs [0023] and [0024] of the present application may be used to define narrower ranges.

In another embodiment of the present disclosure, the method may comprise detecting or measuring a panel of biomarkers comprising Hydrogen Peroxide and Water. In some embodiments, Water may be Water Cluster. In some embodiments of the present disclosure, the panel of biomarkers may comprise Hydrogen Peroxide at about 0.75 times decrease and Water at about 1.4 times increase in comparison with a reference. In some embodiments of the present disclosure, the panel of biomarkers may comprise Hydrogen Peroxide having m/z value being 34.01±1 and Water having m/z value being 38.04±1.

In some embodiments, the method may comprise detecting or measuring a panel of biomarkers comprising Hydrogen Peroxide, Water and 1,3-Butadiene. In some embodiments, Water may be Water Cluster. In some embodiments of the present disclosure, the panel of biomarkers may comprise Hydrogen Peroxide at about 0.75 times decrease, Water at about 1.4 times increase and 1,3-Butadiene at about 1.6 times increase in comparison with a reference. In some embodiments of the present disclosure, the panel of biomarkers may comprise Hydrogen Peroxide having m/z value being 34.01±1, Water having m/z value being 38.04±1 and 1,3-Butadiene having m/z value being 55.05±1.

The panel of biomarkers may comprise any number of biomarkers from the group above (having 10 biomarkers) for example 2, 3, 4, 5, 6, 7, 8, 9, or 10 biomarkers. In some embodiments, the panel of biomarkers further comprises Acetone, Trimethylamine, Isoprene, Carbon Disulfide, 3-Methylpyridine, Benzoic Acid, and Tetradecanoic Acid. In some embodiments, the panel of biomarkers may be selected from the group consisting of Hydrogen Peroxide, Water, 1,3-Butadiene, Acetone, Trimethylamine, Isoprene, Carbon Disulfide, 3-Methylpyridine, Benzoic Acid, and Tetradecanoic Acid. The panel of biomarkers may be detected from a sample obtained from a subject. The panel of biomarkers may be detected from a sample of exhaled breath from a subject. An increase or decrease of the level of each of the biomarker as compared to a control may indicates the presence of a respiratory disease in the subject. In some embodiments of the present disclosure, the method may further comprise applying the levels of biomarkers measured to a decision tree generated using data obtained from subjects without the respiratory disease and subjects with the respiratory disease. In some embodiments, the data obtained from subjects is raw data. In some embodiments, the method may comprise detecting m/z of the biomarkers comprising m/z values being 34.01±1, 38.04±1, 55.05±1, 59.05±1, 60.08±1, 69.08±1, 76.95±1, 94.07±1, 123.04±1, and 229.22±1. In some embodiments, the m/z values being signature m/z values that correspond to Hydrogen Peroxide, Water Cluster, 1,3-Butadiene, Acetone, Trimethylamine, Isoprene, Carbon Disulfide, 3-Methylpyridine, Benzoic Acid, and Tetradecanoic Acid, respectively. In some embodiments, the panel of biomarkers may further comprise the following biomarkers with increase or decrease level of concentration: Acetone (at 1.8 times increase), Trimethylamine (at 1.9 times increase), Isoprene (at 1.1 times increase), Carbon Disulfide (at 2 times increase), 3-Methylpyridine (at 1.8 times increase), Benzoic Acid (at 1.5 times increase), and Tetradecanoic Acid (at 9 times increase). In some embodiments, the increase in the level of each of the biomarkers in comparison with a control or reference may be as follows: Water from about 1.4 times to 100 times, 1,3-Butadiene from about 1.6 times to 100 times, Acetone from about 1.8 times to 100 times, Trimethylamine from about 1.9 times to 100 times, Isoprene from about 1.1 times to 100 times, Carbon Disulfide from about 2 times to 100 times, 3-Methylpyridine from about 1.8 times to 100 times, Benzoic Acid from about 1.5 times to 100 times, and Tetradecanoic Acid from about 9 times to 100 times. It is understood that any range described in paragraphs [0021] and [0022] of the present application may be used to define narrower ranges. In some embodiments, the decrease in the level of each of the biomarkers in comparison with a control or reference may be as follows: Hydrogen Peroxide from about 0.75 times to 0.1 times. It is understood that any range described in paragraphs [0023] and [0024] of the present application may be used to define narrower ranges.

In another embodiment of the present disclosure, the method may comprise detecting or measuring a panel of biomarkers comprising Acetone, 1-Propanol, Nonanal, 3-(Ethylthio)propanal, Butyraldehyde, Acetaldehyde and 3-Hydroxybutyric Acid. The panel of biomarkers may comprise any number and combination of biomarkers from the group above (having 7 biomarkers) for example 2, 3, 4, 5, 6 or 7 biomarkers.

In some embodiments, the panel of biomarkers comprises Acetone and 1-Propanol. In some embodiments of the present disclosure, the panel of biomarkers may comprise Acetone at about 1.4 times increase and 1-Propanol at about 1.2 times increase in comparison with a reference. In some embodiments of the present disclosure, the panel of biomarkers may comprise Acetone having m/z value being 59.05±1 and 1-Propanol having m/z value being 61.01±1.

In some embodiments, the panel of biomarkers comprises Acetone, 1-Propanol and Nonanal. In some embodiments of the present disclosure, the panel of biomarkers may comprise Acetone at about 1.4 times increase, 1-Propanol at about 1.2 times increase and Nonanal at about 2 times increase in comparison with a reference. In some embodiments of the present disclosure, the panel of biomarkers may comprise Acetone having m/z value being 59.05±1, 1-Propanol having m/z value being 61.01±1 and Nonanal having m/z value being 143.24±1.

In some embodiments, the panel of biomarkers comprises Acetone, 1-Propanol, Nonanal and 3-(Ethylthio)propanal. In some embodiments, the panel of biomarkers further comprises Butyraldehyde, Acetaldehyde and 3-Hydroxybutyric Acid. In some embodiments, the panel of biomarkers comprises Acetone, 1-Propanol, Nonanal, 3-(Ethylthio)propanal, Butyraldehyde, and Acetaldehyde. In some embodiments, the panel of biomarkers may be selected from the group consisting of Acetone, 1-Propanol, Nonanal, 3-(Ethylthio)propanal, Butyraldehyde, Acetaldehyde and 3-Hydroxybutyric Acid. The panel of biomarkers may be detected from a sample obtained from a subject. The panel of biomarkers may be detected from a sample of exhaled breath from a subject. An increase or decrease of the level of each of the biomarker as compared to a control may indicates the presence of a respiratory disease in the subject. In some embodiments of the present disclosure, the method may further comprise applying the levels of biomarkers measured to a decision tree generated using data obtained from subjects without the respiratory disease and subjects with the respiratory disease. In some embodiments, the data obtained from subjects is raw data. In some embodiments, the method may further comprise applying the levels of biomarkers measured to a decision tree generated using data obtained from subjects without the respiratory disease and subjects with the respiratory disease. In some embodiments, the method may comprise detecting m/z of the biomarkers comprising m/z values being 59.05±1, 61.10±1, 143.24±1, 119.19±1, 73.06±1, 45.05±1, and 105.11±1. In some embodiments, the m/z values being signature m/z values that correspond to Acetone, 1-Propanol, Nonanal, 3-(Ethylthio)propanal, Acetaldehyde and 3-Hydroxybutyric Acid, respectively. In some embodiments, the panel of biomarkers may further comprise biomarkers with increase or decrease level of concentration: 3-(Ethylthio)propanal (at 2.5 times increase), Butyraldehyde (at 0.5 times decrease), Acetaldehyde (at 4 times increase) and 3-Hydroxybutyric Acid (at 0.5 times decrease). In some embodiments, the increase in the level of each of the biomarkers in comparison with a control or reference may be as follows: Acetone from about 1.4 times to about 100 times, 1-Propanol from about 1.2 times to about 100 times, Nonanal from about 2 times to 100 times, 3-(Ethylthio)propanal from about 2.5 times to 100 times, and Acetaldehyde from about 4 times to 100 times. It is understood that any range described in paragraphs [0021] and [0022] of the present application may be used to define narrower ranges. In some embodiments, the decrease in the level of each of the biomarkers in comparison with a control or reference may be as follows: Butyraldehyde from about 0.5 times to 0.1 times, and 3-Hydroxybutyric Acid from about 0.5 times to about 0.1 times. It is understood that any range described in paragraphs [0023] and [0024] of the present application may be used to define narrower ranges.

In some embodiments, the method of detecting a presence of respiratory disease may further comprise detecting a symptom of respiratory disease. One of ordinary skill in the medical field is trained to recognize whether a subject has a symptom of respiratory disease. Common symptoms of respiratory disease include sneezing, runny nose, sore throat, difficulty breathing, coughing, wheezing, and shortness of breath. The most common symptoms of COVID-19 are fever, dry cough, and tiredness. Less common symptoms of COVID-19 include aches and pains, sore throat, diarrhoea, conjunctivitis, headache, loss of taste or smell, rash on skin, discolouration of fingers or toes. Serious symptoms of COVID-19 include difficulty breathing or shortness of breath, chest pain or pressure and loss of speech or movement. The symptoms of COVID-19 follow from oxidative stress, activation of the immune system, and the specific processes taking place as the COVID-19 infection progresses within the host.

In some embodiments, the method of detecting a presence of respiratory disease may further take into account other variables such as the subject's occupation, demographic data, social data, medical history, vital signs, and time of last meal, drink and smoke. Demographic data and social data may include the subject's nationality, age, gender, residency status, travel history, and whether the subject is or was a smoker. Medical history may include medical information like chronic kidney disease, chronic liver disease, chronic neurological disorder, diabetes, hypertension, heart disease, malignant neoplasm and whether the subject is currently pregnant. Vital signs may include the subject's temperature, blood pressure (BP), oxygen saturation level, height, and weight.

In some embodiments, the method may further comprise administering a pharmaceutical composition to treat the respiratory disease. Examples of types of pharmaceutical compositions to be administered include decongestants, antihistamines, antipyretics, and cough suppressants. Where the respiratory disease identified is caused by a respiratory virus, the pharmaceutical composition administered may be antivirals, including neuraminidase inhibitors, zanamivir, oseltamivir, and peramivir. Where the respiratory disease identified is caused by a respiratory bacterium, the pharmaceutical composition administered may be antibiotics, including amoxycillin and doxycycline. The term “administering” refers to contacting, applying, or providing a composition of the present invention to a subject. In some embodiments, there is provided use of a pharmaceutical composition in the manufacture of a medicament for treating a respiratory disease in a subject, wherein the presence of said respiratory disease is detected in said subject by detecting or measuring a panel of biomarkers disclosed in the present disclosure prior to administering the medicament to the subject. In some embodiments, the detecting or measuring the panel of biomarkers may use the method of detecting and or measuring the panel of biomarkers as disclosed herein.

The term “treating” as used herein may refer to (1) preventing or delaying the appearance of one or more symptoms of the respiratory disease; (2) inhibiting the development of the respiratory disease or one or more symptoms of the respiratory disease; (3) relieving the symptoms of the respiratory disease; and/or (4) inhibiting the causative agent of the respiratory disease if the respiratory disease is caused by a respiratory virus or a respiratory bacterium.

The method as defined herein may comprise obtaining exhaled breath from a subject. Thus, the method described herein may be considered as being a non-invasive method. In one embodiment, the sample may be exhaled breath. In one embodiment, the sample is end-tidal breath. In some embodiments, the sample may be exhaled breath collected using an insulated end-tidal breath sampler which allows a direct path of airway exhalation through an internal heating from 60° C. to 95° C., the sampler allowing real-time breath-gas analysis together with various mass spectrometric techniques of different ionization source. A single exhalation is administered through a tailored tube in which the end-tidal fraction of the breath-gas is buffered, which increases sampling time by an order of magnitude to several seconds, improving signal quality and reducing the total measurement time per test subject. This also reduces the risk of hyperventilation as only one exhalation per minute is required for sampling and the test subject can otherwise maintain a normal breathing pattern. In some embodiments, more than one exhalation (for example two or three exhalations) may be administered through the tailored tube as described above. The length of the end-tidal breath sampler contributes to the buffering and suction pressure to capture the end-tidal fraction of the breath-gas sample. In other embodiments, the sample may be exhaled breath collected using a breath collection machine, such as that disclosed in PCT/SG2020/050361 by the applicant and published on 30 Dec. 2020. In yet another embodiment, the sample may be exhaled breath collected from conventional direct on-line sampling where a subject inhales through their nose and exhales via their mouth through a sampling tube with a one-way valve. In yet another embodiment, the sample may be exhaled breath collected in a bag. The bag may be a tedlar breath bag (or tedlar gas sampling breath) with a valve.

In some embodiments, Volatile Organic Compounds (VOCs) from the breath of a subject may be collected in a sample, e.g., on a filter, either directly or indirectly. The breath samples may be collected in a collection device which may include sorbent tubes, tedlar bags, canisters etc. It can also comprise collecting samples through a real-time breath sampler.

In some embodiments, the breath sample is directly obtained from a subject at or near the laboratory or location where the biological sample will be analysed. In other embodiments, the breath sample may be obtained by a third party and then transferred, e.g., to a separate entity or location for analysis. In other embodiments, the sample may be obtained and tested in the same location using a point-of care test. In these embodiments, said obtaining refers to receiving the sample, e.g., from the patient, from a laboratory, from a doctor's office, from the mail, courier, or post office, etc. In some embodiments, the method may further comprise reporting the determination or test results to the subject, a health care payer, an attending clinician, a pharmacist, a pharmacy benefits manager, or any person that the determination or test results may be of interest. In some embodiments, the sample may be concentrated to facilitate the analysis. In some embodiments, the method of detecting the presence of a respiratory disease disclosed in the present application may be an in vitro method of detecting the presence of a respiratory disease. In some embodiments, the method of detecting the presence of a respiratory disease including a coronavirus infection may have a minimum interference or no interference with biomarkers associated with food or drink consumption, smoking activity, or other diseases. Advantageously, the method disclosed herein may distinguish the presence of the respiratory disease from other diseases. Further advantageously, the method disclosed herein may distinguish the presence of the coronavirus infection from other respiratory diseases. Still advantageously, the method disclosed herein may distinguish the presence of the COVID-19 from other respiratory diseases.

The detection of the biomarkers as defined herein may be using an analytical instrument. Examples of analytical instruments include GC-MS (gas chromatography mass spectrometry), PTR-MS (proton transfer reaction mass spectrometry), PTR-TOF-MS (proton transfer reaction time-of-flight mass spectrometry), SIFT-MS (selected ion flow tube mass spectrometry), and sensor technologies. The detection of the biomarkers may be within the ranges of parts per trillion (ppt) or parts per billion (ppb). In some embodiments, the method of detecting the presence of respiratory diseases may further comprise analysing the sample obtained from the subject using PTR-MS or PTR-TOF-MS. In some embodiments, the method of detecting and measuring the concentrations of biomarkers described herein may use PTR-MS or PTR-TOF-MS.

The method as disclosed above may be undertaken by collecting a breath from a subject and analysing the biomarkers using a mass spectrometer, for example PTR-MS or PTR-TOF-MS. Advantageously, this method may have a short turnaround time of less than 60 seconds from breath sampling to obtaining results (for example less than about 30 seconds, less than about 40 seconds or less than about 50 seconds). The method disclosed herein is also highly efficient, with an accuracy of more than about 85% sensitivity to about 97% sensitivity for example 85%, 86%, 87%, 88%, 89%, 90%, 91%, 92%, 93%, 94%, 95%, 96%, or 97% sensitivity. The accuracy rate may be further increased by using machine learning or artificial intelligence models which are “trained” using data. In some embodiments, the machine learning may be used to address the possible over-fitting, non-normalization and label imbalance problem. In some embodiments, the decision tree-based model may be a known model for example a random forest or XGBoosting.

The method as defined herein may comprise determining a weighted score based on the level of each biomarker in the panel of biomarkers in the sample and comparing it to a weighted score obtained from a control sample. Alternatively, the weighted score on the level of each biomarker in the panel of biomarkers in the sample may be compared to a pre-determined value.

Data analysis algorithm entailing the above conditions with different weightage can be applied to determine the presence of having a respiratory disease.

In some embodiments, the machine learning may comprise constructing a dataset, pre-processing data and building a machine learning model. In some embodiments, the dataset construction may comprise collecting data from the samples obtained from the subjects. The data may comprise sample collection date, spectra of the analytical method used (for example PTR-MS spectra) against time, PCR test result (positive or negative) and other recorded data of the operation of the analytical instrument being used. The dataset may be subsequently divided into a trained dataset and a test dataset. The division may be random and the ratio of the division may be adjusted. In some embodiments, the ratio of the division may be 80% being the trained dataset and 20% being the test dataset. In some embodiments, the ratio of the division may be 75% being the trained dataset and 25% being the test dataset. In some embodiments, the pre-processing data may comprise peak extraction and peak segmentation. In some embodiments, the peak extraction may use a suitable peak detection algorithm. In some embodiments, the intensity of the extracted peaks may be measured in ion counts. In some embodiments, the ion counts may be transformed or converted to concentration (for example in ppb). In some embodiments, the peak segmentation may comprise dividing the spectra or respiratory cycle into three distinct phases: background, exhaling and unclassified phases. The peak segmentation may be performed using median filtering, normalization and quantization on tracing biomarkers throughout the respiratory cycle. In some embodiments, the selected tracing biomarkers may be acetone and isoprene. In some embodiments, the pre-processed data may be obtained by extracting time intervals data, averaging them across the times for background and exhaling phases and calculating the difference between exhaling data and background data to reduce the effect of background VOCs. In some embodiments, the machine learning model may be built to detect the presence of a respiratory disease by PTR-MS breath data. Once the learned machine learning is developed, the same may be used for classification on the testing set. In some embodiments, importance factors for the biomarkers (i.e. VOCs) may be calculated in the model developed. In some embodiments, importance factors for the VOCs may be calculated by averaging the factors across all decision tree in the model. The importance of a feature (for example m/z value) may be calculated by summing the gains of the feature in all trees. Gain is the improvement in accuracy brought by a feature to the branches during the training process.

In one embodiment, there is provided a method comprising the steps of a) detecting and measuring a panel of biomarkers comprising Water, Methanol, 1,3-Butadiene, Acetone, Isoprene, Isobutyronitrile, 3-Methylpyridine, Pentanoic Acid, m-Cresol or p-Cresol, N,N-Dimethylaniline, and n-Decane in a sample obtained from the subject, b) determining whether the subject has a respiratory disease, and c) administering a pharmaceutical composition to the subject found to have a respiratory disease. In some embodiments, the method described above is a method of detecting and treating a respiratory disease in a subject.

In one embodiment, there is provided a method comprising the steps of a) detecting and measuring a panel of biomarkers comprising Butanol, Isoprene, Acetone, Cycloheptene, and Sesquiterpene in a sample obtained from the subject, b) determining whether the subject has a respiratory disease, and c) administering a pharmaceutical composition to the subject found to have a respiratory disease. In some embodiments, the method described above is a method of detecting and treating a respiratory disease in a subject.

In one embodiment, there is provided a method comprising the steps of a) detecting and measuring a panel of biomarkers comprising 1,3-Butadiene, Acetone, Isoprene, Methacrolein, Butanal, Propanoic Acid, Aniline, Furfural, 1-Octene, 6-Methyl-5-Hepten-2-one, 1-Decene, Dodecane, β-Damascenone, Sesquiterpene, and Tetradecanoic Acid in a sample obtained from the subject, b) determining whether the subject has a respiratory disease, and c) administering a pharmaceutical composition to the subject found to have a respiratory disease. In some embodiments, the method described above is a method of detecting and treating a respiratory disease in a subject.

In one embodiment, there is provided a method comprising the steps of a) detecting and measuring a panel of biomarkers comprising Hydrogen Peroxide, Water, 1,3-Butadiene, Acetone, Trimethylamine, Isoprene, Carbon Disulfide, 3-Methylpyridine, Benzoic Acid, and Tetradecanoic Acid in a sample obtained from the subject, b) determining whether the subject has a respiratory disease, and c) administering a pharmaceutical composition to the subject found to have a respiratory disease. In some embodiments, the method described above is a method of detecting and treating a respiratory disease in a subject.

In one embodiment, there is provided a method comprising the steps of a) detecting and measuring a panel of biomarkers comprising Acetone, 1-Propanol, Nonanal, 3-(Ethylthio)propanal, Butyraldehyde, Acetaldehyde, and 3-Hydroxybutyric Acid in a sample obtained from the subject, b) determining whether the subject has a respiratory disease, and c) administering a pharmaceutical composition to the subject found to have a respiratory disease.

In some embodiments, the method described above is a method of detecting and treating a respiratory disease in a subject.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions, and compounds referred to or indicated in this specification, individually or collectively, and any and all combinations of any two or more of said steps or features.

Throughout this specification and the statements which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Certain embodiments of the invention will now be described with reference to the following examples which are intended for the purpose of illustration only and are not intended to limit the scope of the generality hereinbefore described.

### EXAMPLES

### Study Design

The research was carried out at two sites in two countries: Singapore as a First Clinical Site, and Dubai as a Second Clinical Site.

### Breath Collection Procedures

Breath samples were collected through two methods: (i) direct exhalation into an analysis equipment; and (ii) exhalation into a bag before transfer for analysis. For breath collection through direct exhalation into an analysis equipment, the breath samples were collected using a Buffered End-Tidal Breath Sampling Inlet (BET) (purchased from Ionicon, Austria). A subject administers a single exhalation through a tailored tube in which the end-tidal fraction of the breath-gas sample is buffered.

For breath collection through exhalation into a breath bag before transfer for analysis, subjects exhaled into a commercially available tedlar sample bag until the tedlar sample bag was fully inflated.

### Data Collection

Several data variables were collected from the recruited subjects for analysis. The data variables collected from the recruited subjects are presented below in Table 1.

TABLE 1
Data Variables obtained from Recruited Subjects

| | |
|---|---|
| Participant study ID | |
| Date of COVID-19 screening | |
| Indication for COVID-19 | Contact with confirmed case/Occupational |
| screening | exposure/Travel |
| Demographic data/social | Nationality |
| data | Age |
| | Gender |
| | Residency Status/Tourist |
| | Travel History (travel dates and country) |
| | Current smoking (Y/N) (if yes, pack-year |
| | of smoking) |
| | Past smoking (Y/N) (if yes, pack-year of |
| | smoking) |
| Healthcare workers only | Working in a COVID-19 ward/isolation |
| | center (Y/N) |
| | If yes: please state duration; |
| | Setting of work: ICU/General |
| | ward/Isolation Center/Other |
| Medical history | Chronic kidney disease |
| | Chronic liver disease |
| | Chronic neurological disorder |
| | Diabetes |
| | Hypertension |
| | Heart disease |
| | Malignant neoplasm |
| | Pregnant (if yes; state gestational |
| | age in weeks) |
| COVID-19 symptoms | Yes/NO (if yes, specify symptoms) |
| (For each symptom present, | History of self-reported feverishness |
| the date of onset will be | or measured fever of ≥38° C. |
| noted) | Cough |
| | Sore throat |
| | Shortness of breath |
| | Myalgia/arthralgia |
| | Eye pain |
| | Loss of sense of smell |
| | Loss of sense of taste |
| | Other |
| Vital signs | Temperature |
| | BP |
| | Oxygen saturation (where taken) |
| | Height/Weight |
| Time of last meal, drink | |
| and smoke (if applicable) | |

### Proton-Transfer-Reaction Mass Spectrometry (PTR-MS) Measurement

Measurement of breath samples was carried out with Proton-Transfer-Reaction Mass Spectrometry (PTR-MS) (Model TOF500 (First Clinical Site), TOF1000 (Second Clinical Site), or TOF1000X, Ionicon Analytik GmbH, Innsbruck, Austria). Where a breath sample was collected through direct exhalation, the sampling inlet was connected to the PTR-MS to transfer the breath sample directly into the PTR-MS in real time for analysis. This method effectively avoided sample absorption, storage, and transportation, thereby minimizing sample loss and contamination. The method also advantageously enables fast on-spot detection which allows point-of-care diagnosis. Where a breath sample was collected through exhalation into a bag, the tedlar sample bag was connected to the PTR-MS to transfer the breath sample into the PTR-MS for analysis.

The PTR-MS measures the concentration of a few hundred biomarkers or VOCs, some of which are disease biomarkers and will be singled out for data analysis. The PTR-MS instrument comprises an ionization section and a detection section. During the ionization process, the instrument forms protonated water ions (H₃O⁺) by a hollow cathode discharge in the ion source. These H₃O⁺ ions are then introduced into the drift tube by an electric drift field, where they chemically ionize the biomarkers including volatile organic compounds (VOC) in breath samples via proton-transfer reaction (PTR). Only the biomarkers or VOCs with higher proton affinity (PA) value than that of H₂O molecules will be ionized by H₃O⁺ and proceed to the detection section. These ionized VOCs are extracted by the electric field towards the time-of-flight mass spectrometer (TOF-MS) to be differentiated and detected with respect to their mass-to-charge ratio (m/z). The drift tube voltage was set at 600 V and the drift tube pressure was set at 2.3 mbar. The reduced electric field (or E/N ratio) was 139 Townsend (Td). The sampling line and buffer tube were kept at 70° C. The only difference between PTR-MS TOF500, PTR-MS TOF1000 and PTR-MS TOF1000X instruments is the sensitivity or limit of detection (LOD), which was all within the ranges of parts per trillion (ppt). As the concentrations of VOCs detected in the breath were in the parts per billion (ppb) range, which is significantly higher than the reported LOD, the results obtained from the different PTR-MS machines were assumed to be comparable. Furthermore, as the PTR-MS TOF500, PTR-MS TOF1000, and PTR-MS TOF1000X use the same technology, it was assumed that the same results would be obtained from all three instruments.

For each subject, a new data file was recorded by acquiring a 10-second run of background measurement before the first exhalation or commencement of transfer of sample from the tedlar sample bag. Mass calibration of the data was carried out by using masses 21.0226 (H₃O⁺ isotope) and 60.049 (Acetone isotope), or 203.943 (diiodobenzene fragment) and was calibration was carried out once every 60 seconds.

### Processing of Breath Data

Raw data was processed using viewer software to perform mass calibration and to calculate peaks present in the data. A trace analyser was used to identify background air and exhaled breath. Datapoints from exhaled breath were selected and averaged for each subject. Molecules with concentrations lower in the breath than in the background were excluded from subsequent data analysis.

### Logistic Regression Model

The biomarkers detected were used to construct a ROC (receiver operating characteristics) curve based on a logistic regression model.

### Decision Tree

A preliminary screening of potential biomarkers was carried out using differential analysis to identify the mass-to-charge (m/z) ratio of molecules that may be used to distinguish subjects with and without COVID-19 by determining m/z ratio values with significant differences in concentration between subjects with and without COVID-19. A Shapiro-Wilk test was first carried out to determine if the concentration of molecules followed a normal distribution. If the Shapiro-Wilk test revealed that the concentration of molecules did not follow a normal distribution, a non-parametric Wilcoxon test would be used to carry out the differential analysis to determine molecules of interest with concentrations that were significantly different between subjects with and without COVID-19.

A decision tree model was then built to classify subjects into COVID-19 positive and negative groups was built using the molecules of interest identified from the Wilcoxon test. The complexity of the tree and minimum number of samples per node were limited to enhance the generalization and classification performance, and to obtain an optimal decision tree. Preferably, the decision tree generated has at least 85% sensitivity (for example 85%, 86%, 87%, 88%, 89%, 90%, 91%, 92%, 93%, 94%, 95%, 96%, 97%, 98%, 99%, or 100%) and 95% specificity (for example 95%, 96%, 97%, 98%, 99%, or 100%).

All the samples obtained in a group were used to generate a decision tree. For a given molecule (or biomarker) a containing n samples of different concentrations, the values were arranged from smallest to largest and presented as {a₁, a₂, a₃, . . . , aₙ}. Different concentration threshold potentials t were then calculated using equation (ii) below in order to classify the samples:

$${T}_{a} = \{ \frac{{a}_{i} + {a}_{i + 1}}{2} \vert 1 \leq i \leq n - 1 \} \qquad ( ii )$$

The midpoint

$${t}_{i} = \frac{{a}_{i} + {a}_{i + 1}}{2}$$

of an interval [aᵢ, aᵢ₊₁] was used as the division point for the concentration threshold potential to divide the data into two subsets, Dₜ⁻ and Dₜ⁺. Dₜ⁻ represented the set of samples with molecule concentrations less than t, while Dₜ⁺ represented the set of samples with molecule concentrations greater than t.

The information gain for a molecule whose concentration threshold potential was less than t was then calculated using equation (iii):

$$Gain ( D , a , t ) = Ent ( D ) - [ \frac{{D}_{t}^{-}}{D} Ent ( {D}_{t}^{-} ) + \frac{{D}_{t}^{+}}{D} Ent ( {D}_{t}^{+} ) ] \qquad ( iii )$$

Ent(D) is called the information entropy and is one of the most commonly used metrics to measure the purity of a sample set. The smaller the value of Ent(D), the higher the purity of D will be. Assuming that the proportion of positive and negative cases in the sample set D were p⁺ and p⁻ respectively, then the information entropy is given by either equation (iv) or (v):

$$Ent ( D ) = - ( {p}^{+} {log}_{2} {p}^{+} + {p}^{-} {log}_{2} {p}^{-} ) \qquad ( iv )$$

$$Ent ( D ) = - ( {p}^{+} {p}^{+} + {p}^{-} {p}^{-} ) \qquad ( v )$$

In general, the greater the information gain of a molecule, the greater the “purity gain” will be when that molecule is used as a feature to split the dataset, i.e., there is a gain of information that splitting in that particular way on that biomarker gives a particular output. On the other hand, if a split that does not provide information gain is chosen, there would not be additional knowledge on whether a particular node had any influence in classify the data. Therefore, different concentration threshold potentials t were used to obtain multiple information gains for a particular molecule and the largest information gain and its corresponding concentration threshold potential was used as the criterion for the splitting. Using this concept, the maximum information gain of all molecules was found and the molecule with the highest information gain was used as the first node of the tree (i.e., the root node).

Table 2 illustrates a table showing examples of concentrations of two biomarkers (in ppb). The information gain for biomarker 1 is calculated to be 0.262 and the information gain for biomarker 2 is calculated to be 0.349. Based on the concentrations in Table 2 and the information gain calculated, biomarker 2 would be used as the first feature or node for splitting as the information gain is higher and the corresponding value of concentration threshold potential is determined to be 0.381.

TABLE 2
Example of Biomarker Concentrations

| | | Biomarker 1 | Biomarker 2 |
|---|---|---|---|
| | Samples | Concentration | Concentration |
| Positive | 1 | 0.697 | 0.460 |
| Cases | 2 | 0.774 | 0.376 |
| | 3 | 0.634 | 0.264 |
| | 4 | 0.608 | 0.318 |
| | 5 | 0.556 | 0.215 |
| | 6 | 0.403 | 0.237 |
| | 7 | 0.481 | 0.149 |
| | 8 | 0.437 | 0.211 |
| Negative | 9 | 0.666 | 0.091 |
| Cases | 10 | 0.243 | 0.267 |
| | 11 | 0.245 | 0.057 |
| | 12 | 0.343 | 0.099 |
| | 13 | 0.639 | 0.161 |
| | 14 | 0.657 | 0.198 |
| | 15 | 0.360 | 0.370 |
| | 16 | 0.593 | 0.042 |
| | 17 | 0.719 | 0.103 |

Once the first molecule (root node) was identified, its corresponding concentration threshold potential for division was determined. The two subsets of the split, D⁻ and D⁺ were identified and the process was repeated for each intermediate node until all samples had been split into the leaf nodes.

Once the decision tree was generated, the decision tree was “pruned”. This is because the structure of the tree may be so complex that the model may be over-fitted, i.e., it performs well on a training set but poorly on a test set. “Pruning” using a cross-validation method was carried out to prevent overfitting. With the cross-validation method, all the samples were divided into ten equal parts: nine parts were used as the training set, while the remaining part was used as the test set. This modelling process was carried out ten times and the average accuracy of the ten times on the test set was used as the evaluation criterion. If there was overfitting, a low accuracy will be obtained on the test set, the node will be removed from the decision tree starting from the bottom of the decision tree to the top until the best performance was obtained on the test set.

### Verification of Results

The presence or absence of infection with SARS-CoV-2 was confirmed using commercially available SARS-CoV-2 polymerase chain reaction (PCR) tests for each subject. For example, viral RNA may be extracted using a EZ1 DSP Virus Kit (Qiagen, Hilden, Germany), while SARS-CoV-2 RT-PCR for the detection of three gene targets (N, E and RdRp genes) may be carried out using the Allplex™ 2019-nCoV Assay (Seegene, Seoul, South Korea).

### Results of Analysis of Data

Group 1: Analysis of Data from Direct Exhalation and Breath Bag Samples from First Clinical Site Up to a First Cut-Off Date

Table 3 illustrates a first list of biomarkers identified to be correlated to a presence of COVID-19 based on results obtained from direct exhalation and breath bag samples at the first clinical site up to a first cut-off date.

TABLE 3
List of Biomarkers Identified at First Clinical Site

| S/N | m/z | Biomarker | (+) Samples (ppb) | (−) Samples (ppb) |
|---|---|---|---|---|
| 1 | 75.12 | Butanol | 0.4~3.7 | 0.2~1.3 |
| 2 | 69.07 | Isoprene | 48~185 | 46~102 |
| 3 | 59.05 | Acetone | 540~1620 | 480~980 |
| 4 | 97.10 | Cycloheptene | 0.04~0.5 | 0.06~0.7 |
| 5 | 205.20 | Sesquiterpene | 0~0.3 | 0~0.2 |

The biomarkers identified were validated against a gold standard reference method of PCR assay, and the cut-off point was determined. The results were determined using a receiver operating characteristics (ROC) curve constructed using the combined identified biomarkers based on a logistic regression model. The formula of the regression model is equation (i). A sample with a y-value higher than a defined cut-off value was classified as positive for COVID-19 infection. For example, if the defined cut-off value, or COP, was 0.2, any samples with a y-value higher than 0.2 was classified as COVID-19 positive. As can be seen from Table 3, the samples that were tested positive generally show increase in the concentration level of biomarkers compared to the samples being tested negative (Butanol with about up to 3 times increase, Isoprene having an increase of about 1.8 times, Acetone with about up to 1.7 times increase, Cycloheptene with about up to 1.5 times increase, and Sesquiterpene with about up to 1.5 times increase).

Group 2: Analysis of Data from Direct Exhalation and Breath Bag Samples from First Clinical Site Up to a Second Cut-Off Date

Table 4 illustrates a second list of biomarkers identified to be correlated to a presence of COVID-19 based on results obtained from direct exhalation and breath bag samples at the first clinical site up to a second cut-off date. As can be seen from Table 4, the samples that were tested positive show an increase in the concentration level for some of the biomarkers compared to the samples being tested negative (1,3-Butadiene with about up to 1.5 times increase, Acetone having an increase of about 1.7 times, Isoprene with about up to 1.8 times increase, Propanoic Acid with about up to 4 times increase, Aniline with about up to 1.8 times increase, 1-Octene having an increase of about 1.6 times, 1-Decene having an increase of 8 times, Dodecane having an increase of about 5 times, β-Damascenone having an increase of about 17 times, Sesquiterpene having an increase of about 1.3 times, and Tetradecanoic Acid having an increase of about 1.8 times). Further, as can be seen from Table 4, the concentration of some biomarkers in the positive tested sample is lower than that in the negative tested sample (Methacrolein with a decrease of about 0.6 times, Butanal with a decrease of about 0.5 times, Furfural having a decrease of about 0.7 times, and 6-Methyl-5-Hepten-2-one having a decrease of about 0.16 times).

TABLE 4
Second List of Biomarkers Identified at First Clinical Site

| | | | (+) Samples | (−) Samples |
|---|---|---|---|---|
| S/N | m/z | Biomarker | (ppb) | (ppb) |
| 1 | 55.05 | 1,3-Butadiene | 5~34 | 4~22 |
| 2 | 59.05 | Acetone | 610~1520 | 440~890 |
| 3 | 69.08 | Isoprene | 54~177 | 46~99 |
| 4 | 71.05 | Methacrolein | 0.3~1.7 | 0.5~2.3 |
| 5 | 73.06 | Butanal | 0~0.4 | 0~0.7 |
| 6 | 75.04 | Propanoic Acid | 0.4~3.7 | 0.1~3.3 |
| 7 | 94.07 | Aniline | 0~0.7 | 0~0.4 |
| 8 | 97.03 | Furfural | 0.06~0.6 | 0.06~0.8 |
| 9 | 113.13 | 1-Octene | 0~0.8 | 0~0.5 |
| 10 | 127.11 | 6-Methyl-5-Hepten-2-one | 0~0.01 | 0~0.06 |
| 11 | 141.16 | 1-Decene | 0~0.03 | 0~0.004 |
| 12 | 171.21 | Dodecane | 0~0.005 | 0~0.001 |
| 13 | 191.14 | β-Damascenone | 0~0.01 | 0~0.0006 |
| 14 | 205.20 | Sesquiterpene | 0~0.25 | 0~0.2 |
| 15 | 229.22 | Tetradecanoic Acid | 0~0.007 | 0~0.004 |

### Group 3: Analysis of Data Up to a Third Cut-Off Date

Table 5 illustrates a third list of biomarkers identified to be correlated to a presence of COVID-19. As can be seen from Table 5, the samples that were tested positive show increase in the concentration level for some of the biomarkers compared to the samples being tested negative (Water Cluster having an increase of about 1.4 times, 1,3-Butadiene with an increase of about 1.6 times, Acetone having an increase of about 1.8 times, Trimethylamine having an increase of about 1.9 times, Isoprene having an increase of about 1.1 times, Carbon Disulfide having an increase of about 2 times, 3-Methylpyridine having an increase of about 1.8 times, Benzoic Acid having an increase of about 1.5, and Tetradecanoic Acid having an increase of about 9). Further, as can be seen from Table 5, the concentration of one biomarker i.e. Hydrogen Peroxide in the positive tested sample is lower than that in the negative tested sample (a decrease of about 0.75 times).

TABLE 5
Third List of Biomarkers Identified

| | | | (+) Samples | (−) Samples |
|---|---|---|---|---|
| S/N | m/z | Biomarker | (ppb) | (ppb) |
| 1 | 34.01 | Hydrogen Peroxide | 0~0.9 | 0~1.2 |
| 2 | 38.04 | Water Cluster | 64~130 | 52~96 |
| 3 | 55.05 | 1,3-Butadiene | 5~34 | 4~22 |
| 4 | 59.05 | Acetone | 710~1490 | 390~830 |
| 5 | 60.08 | Trimethylamine | 20~82 | 15~43 |
| 6 | 69.08 | Isoprene | 48~115 | 46~102 |
| 7 | 76.95 | Carbon Disulfide | 0~0.02 | 0~0.01 |
| 8 | 94.07 | 3-Methylpyridine | 0~0.7 | 0~0.4 |
| 9 | 123.04 | Benzoic Acid | 0~0.6 | 0~0.4 |
| 10 | 229.22 | Tetradecanoic Acid | 0~0.008 | 0~0.0009 |

Group 4: Analysis of Data from all Direct Exhalation Samples from Both Clinical Sites

Of the total number of subjects recruited, samples were obtained through direct exhalation into the PTR-MS from 480 subjects: 176 subjects were recruited from the First Clinical Site and 304 subjects were recruited from the Second Clinical Site. Among the 480 subjects within this first group, 116 subjects were COVID-19 positive while 364 subjects were controls without COVID-19. Among the 116 COVID-19 positive subjects, 18 were asymptomatic when they visited the clinic and the clinical information of 2 subjects from the First Clinical Site were still pending when data analysis was carried out. Clinical information of the subjects recruited are presented below in Table 6.

TABLE 6
Clinical Information of Subjects of Group 4

| | First Clinical Site | | Second Clinical Site | | Total | |
|---|---|---|---|---|---|---|
| | COVID-19 | Control | COVID-19 | Control | COVID-19 | Control |
| Total Number | 17 | 159 | 99 | 205 | 116 | 364 |
| Gender (M/F) | 17/0 | 137/22 | 48/51 | 122/83 | 65/51 | 259/105 |
| Age (Mean ± sdv) | 32 ± 9 | 36 ± 9 | 37 ± 11 | 37 ± 13 | 37 ± 11 | 37 ± 12 |
| Smoking History | | | | | | |
| Non-smoker | 11 | 97 | 91 | 159 | 102 | 256 |
| Current smoker | 4 | 29 | 8 | 44 | 12 | 73 |
| Ex-smoker | 2 | 33 | 0 | 2 | 2 | 35 |

The Shapiro-wilk test carried out showed that the concentration of molecules obtained were not of a normal distribution. Thus, a Wilcoxon test was used to carry out differential analysis to determine which molecules had significantly different concentrations between subjects with and without COVID-19. The Wilcoxon test carried out identified 18 molecules that had significant differences in concentration between subjects with COVID-19 and subjects without COVID-19.

### Generation of Decision Tree

FIG. 1 is a schematic illustration of a decision tree 100 generated using data from samples collected through direct exhalation, in accordance with embodiments of the present disclosure. All the samples obtained in this group were used to generate decision tree 100. The complexity of the tree was set at 0.005 and minimum number of samples per node was set to 8 samples.

Decision tree 100 comprises multiple nodes. Decision tree 100 starts with a root node 104, goes through multiple intermediate nodes 108, which ends with terminal nodes 112. Each node 104 and 112 in the decision tree 100 makes decisions according to the concentration of a molecule 116 with an m/z value stated underneath the node. In other words, samples are sorted into the next node based on the concentration of a molecule 116 with an m/z value stated underneath the node. The left side of the tree was divided into subjects who were COVID-19 positive, while the right side of the tree was divided into subjects who were COVID-19 negative. Each node comprises three numbers: first number 120, second number 124, and third number 128 as shown in FIG. 1. First number 120 represents the category or population represented in the node. First number 120 may have a value of 0 if there is a higher proportion of subjects who are COVID-19 negative in the node, and a value of 1 if there is a higher proportion of subjects who are COVID-19 positive in the node. Second number 124 represents the proportion of subjects who are COVID-19 positive in the node. Third number 128 represents the percentage of total samples in the node. Root node 104 has a first number 120 with a value of 0 indicating that there is a higher proportion of subjects who are COVID-19 negative in root node 104, a second number 124 with a value of 0.29 indicating that 29% of the samples in root node 104 are COVID-19 positive, and a third number 128 with a value of 100% indicating that 100% of the total sample population is within this node. In addition, root node 104 makes decisions according to the concentration of a molecule 116 with an m/z value of 143.90. Where a sample within root node 104 has the concentration of molecule 116 with an m/z value of 143.90 is more than or equal to 0.085 ppb, it is split or sorted into intermediate node 108a on the left. Where a sample within root node 104 has the concentration of molecule 116 with an m/z value of 143 is less than 0.085 ppb, it is split or sorted into intermediate node 108b on the right.

11 m/z ratios were found to be sufficient to build a decision tree having a sensitivity of 85.34% and specificity of 96.98%, respectively. The 11 m/z values used were ‘m038’, ‘m051’, ‘m055’, ‘m059’, ‘m069’, ‘m070’, ‘m094, ‘m103, ‘m109’, ‘m122’ and ‘m143’.

### Identification of Biomarkers

Table 7 illustrates a list of biomarkers identified to be correlated to a presence of COVID based on data from samples collected through direct exhalation. Possible compounds for each mass-to-charge ratio were identified from an internal database of molecules. A literature search was then carried out to determine the biological relevance of each identified compound. As can be seen from Table 7, the samples that were tested positive show increase in the concentration level for some of the biomarkers compared to the samples being tested negative (Water (Cluster) having an increase of about 1.3 times, Methanol (Water Cluster) with an increase of about 2.6 times, 1,3-Butadiene having an increase of about 2.5 times, Acetone having an increase of about 1.5 times, Isoprene having an increase of about 1.1 times, Isobutyronitrile having an increase of about 1.3 times, 3-Methylpyridine having an increase of about 1.7 times, m-Cresol or p-Cresol having an increase of about 2.5 times and N,N-Dimethylaniline having an increase of about 1.3 times). Further, as can be seen from Table 7, the concentration of some biomarkers in the positive tested sample is lower than that in the negative tested sample (Pentanoic Acid having a decrease of about 0.3 times and n-Decane with a decrease of about 0.7 times).

TABLE 7
Fourth List of Biomarkers Identified

| | | | CAS | (+) Samples | (−) Samples |
|---|---|---|---|---|---|
| S/N | m/z | Biomarker | Number | (ppb) | (ppb) |
| 1 | 38.04 | Water (Cluster) | 7732-18-5 | 42~81 | 34~65 |
| 2 | 51.04 | Methanol (Water | 67-56-1 | 10~23 | 7~9 |
| | | Cluster) | | | |
| 3 | 55.05 | 1,3-Butadiene | 106-99-0 | 3~17 | 3~7 |
| 4 | 59.05 | Acetone | 67-64-1 | 460~1070 | 350~710 |
| 5 | 69.08 | Isoprene | 78-79-5 | 36~74 | 35~67 |
| 6 | 70.07 | Isobutyronitrile | 78-82-0 | 2~4 | 2~3 |
| 7 | 94.07 | 3-Methylpyridine | 108-99-6 | 0~0.5 | 0~0.3 |
| 8 | 103.08 | Pentanoic Acid | 109-52-4 | 0~0.06 | 0~0.2 |
| 9 | 109.07 | m-Cresol or | 108-39-4 | 0~5 | 0~2 |
| | | p-Cresol | 106-44-5 | | |
| 10 | 121.96 | N,N- | 121-69-7 | 0~0.2 | 0~0.15 |
| | | Dimethylaniline | | | |
| 11 | 143.18 | n-Decane | 124-18-5 | 0~0.1 | 0~0.14 |

The compound with m/z value of 38 may be water (cluster), which has an m/z value of 38.04. Water vapour is present in exhaled breath as the inner lining of the lungs is wet and the air in the lung will pick up the water molecules from these surfaces as we exhale. Water in exhaled air may be used as a non-invasive means to measure airway inflammation.

The compound with m/z value of 51 may be Methanol (water cluster), which has an m/z value of 51.04. There are many sources of physiological Methanol in humans. The main source of exogenous Methanol in healthy human are fruits, vegetables, and alcoholic beverages. Anaerobic fermentation by gut bacteria and the transformation of S-adenosyl methionine (SAM) to Methanol by certain metabolic processes are the two other sources of physiological Methanol. Regardless of the source that Methanol is derived in the body, the human body will eventually keep the Methanol level in the body at a low physiological level via metabolic clearance mechanism (i.e., oxidation to formaldehyde and eventually to formic acid which can then be oxidised to water and carbon dioxide) or directly excreted in the urine and exhaled breath. If there are any changes in the human body that leads to a breakdown in any of the metabolic clearance mechanism, the amount of physiological Methanol in the body will increase and hence the human body will remove more Methanol by physiological means (i.e., urine and breath) in order to maintain a low physiological Methanol level. High concentrations of Methanol have been found in the breath in patients with lung cancer.

The compound with m/z value 55 may be 1,3-Butadiene, which has an m/z value of 55.05. The main source of exposure to 1,3-Butadiene is from cigarette smoke and thus inhalation of the chemical is the main route of uptake. In the body, 1,3-Butadiene is broken down by the enzymes in the liver and the metabolic products are then excreted in the urine. Thus, 1,3-Butadiene in the breath has been mainly used as a biomarker for smoking. It has also been used to detect patients with liver diseases, namely non-alcoholic and alcoholic fatty liver disease, and liver cirrhosis.

The compound with m/z value of 59 may be Acetone, which has an m/z value of 59.05. The major source of physiological Acetone in the body is from the decarboxylation of acetoacetate. This reaction can occur spontaneously or can be catalysed by the enzyme acetoacetate decarboxylase. Acetoacetate and the resulting Acetone are produced during the beta-oxidation of fatty acid, glycolysis (breaking down of glucose) and metabolism of amino acids and the rate limiting step in the synthesis cycle is the formation of HMG-COA. As the formation of Acetone involves many different pathways for various substrates, a high level of Acetone is often associated with many diseases. High concentrations of Acetone found in the breath have been used as a biomarker for several diseases such as lung cancer, colorectal cancer, diabetes, non-alcoholic fatty liver disease in children, liver disease, and cystic fibrosis.

The compound with m/z value of 69 may be Isoprene, which has an m/z value of 69.08. The primary source of Isoprene in human is attributed to the mevalonate pathway of cholesterol biosynthesis. Originating from acetyl-CoA, mevalonate is transformed into dimethylallyl pyrophosphate (DMPP). Subsequently, Isoprene can be derived from DMPP via an acidic decomposition process. Isoprene found in breath had been used as biomarker for lung cancer, chronic kidney disease, non-alcoholic fatty liver disease in children, chronic liver disease, liver cirrhosis, advanced fibrosis in patients with chronic liver disease, and monitoring of high blood cholesterol level.

The compound with m/z value of 70 may be Isobutyronitrile, which has an m/z value of 70.07. Isobutyronitrile is a compound added into cigarettes and petrol and thus enters the body via inhalation when smoking cigarettes. Isobutyronitrile has been used as a breath biomarker to identify smokers.

The compound with m/z value of 94 may be 3-Methylpyridine, which has an m/z value of 94.07. The main source of absorption of 3-Methylpyridine into the body is via ingestion as the chemical is used in flavouring agents and can be found in tea and oranges. 3-Methylpyridine had previously been detected at elevated levels in the breath of smokers, breast cancer patients, and patients with periodontitis.

The compound with m/z value of 103 may be Pentanoic Acid, which has an m/z value of 103.08. Pentanoic Acid enters the body mainly via ingestion as the compound is found naturally in vegetables, food additives and pharmaceuticals. Another route of entry is via inhalation as the compound is also mainly used as perfumes and cosmetics. Pentanoic Acid has been used as a breath biomarker in lung cancer, chronic obstructive pulmonary disease, smokers, colorectal cancer, esophagogastric cancer, and Crohn's disease.

The compound with m/z value of 109 may be m-cresol or p-cresol, which has an m/z value of 109.07. Absorption of mixed cresol into the body can occur via inhalation and ingestion as the ambient air contains low level of cresols from car exhaust, power plants, and cigarettes. Mixed cresols are also found in food products such as tomatoes, ketchup, cheeses, butter, bacon, red wines, roasted coffee, and black tea. Thus, cresols have been found in the breath of smokers and have been used as biomarkers to differentiate patients with asthma and renal disease.

The compound with m/z value of 122 may be N,N-Dimethylaniline, which has an m/z value of 121.96. N,N-Dimethylaniline is present in certain antibiotics (penicillin and cephalosporin) as an impurity and hence subjects may have been exposed to it through their use. This chemical had been used as a breath biomarker to detect emphysema.

The compound with m/z value of 143 may be n-Decane, which has an m/z value of 143.18. Absorption of n-Decane occurs mainly through inhalation as the volatile compound is mainly found in fuel, crude oil, and natural gas. Humans may also come into contact with the compound when using gasoline products, eating fish and shellfishes, or even drinking water. This compound has been used as a breath biomarker in lung cancer, asthma, tuberculosis, and in smokers.

Table 8 illustrates a table summarizing the association of the identified molecules with COVID-19. The most common symptoms of COVID-19 are fever, dry cough, and tiredness.

Some less common symptoms include aches and pains, sore throat, diarrhoea, conjunctivitis, headache, loss of taste or smell, rash on skin, discolouration of fingers or toes. Examples of serious symptoms of the disease include difficulty breathing or shortness of breath, chest pain or pressure and loss of speech or movement. These representations of the SARS-CoV-2 virus infection follow from the oxidative stress, activation of the immune system, and the specific processes taking place as the COVID-19 infection progresses within the host.

A description of how the COVID-19 infection progresses is as follows: Once in the body through respiratory system, the SARS-CoV-2 virus attacks angiotensin converting enzyme-2 (ACE-2)-containing cells on the respiratory tract and starts replicating. Apoptotic cells are wiped out, filling the airways, and transporting the virus deeper into the body down towards the lungs. As the infection progresses, the lungs become clogged with dead cells and fluid, making breathing difficult. The immune system is activated; this causes inflammation and fever. At this stage, cellular immunity develops by activating T-cells targeting the generated antigens to neutralize them. Cytokines are released to regulate the cell-level immunity processes. In severe states of COVID-19, cytokine storm is activated. During a cytokine storm, uncontrolled amounts of cytokines are released by the immune system. When it happens, people are more susceptible to infectious virus. Storms can affect other organs besides the lungs, especially in people with chronic illness.

As a consequence, each phase of COVID-19 infection involves specific metabolic reactions, generating biomarker profiles that are specific to the SARS-CoV-2 manifestations in exhaled human breath.

TABLE 8
Correlation between Identified Molecules with COVID-19

| | Molecules | | Disease and its Symptoms |
|---|---|---|---|
| | (# S/N of Molecule | Symptoms of | that are Similar to |
| S/N | in Table 7) | COVID-19 | COVID-19 |
| 1 | #2 Methanol | Cough | Lung Cancer: |
| | #4 Acetone | | Cough |
| | #5 Isoprene | Tiredness | Ache or pain when |
| | #8 Pentanoic acid | Aches and pains | breathing or coughing |
| | #11 n-Decane | Shortness of breath | Persistent breathlessness |
| | | | Persistent tiredness or lack |
| | | | of energy |
| 2 | #11 n-Decane | Fever | Tuberculosis: |
| | | Cough | Fever |
| | | | Cough |
| 3 | #8 Pentanoic acid | Cough | Chronic Obstructive |
| | | Shortness of breath | Pulmonary Disease: |
| | | | Cough |
| | | | Shortness of breath |
| 4 | #9 m-Cresol or p-Cresol | Cough | Asthma: |
| | | Tiredness | Coughing |
| | | | Chest tightness |
| | #11 n-Decane | Shortness of breath | Drowsiness, confusion, |
| | | Chest pain | exhaustion or dizziness |
| | | | Fainting |
| | | | Persistent breathlessness |
| 5 | #10 N,N-Dimethylaniline | Cough | Emphysema: |
| | | Shortness of breath | Cough |
| | | | Shortness of breath |
| 6 | #2 Water | Cough | Airway inflammation: |
| | | Chest pain | Coughing |
| | | Shortness of breath | Shortness of breath |
| | | | Breathing difficulties |
| 7 | #3 1,3-Butadiene | Tiredness | Liver Disease: |
| | #4 Acetone | Rash on skin | Weakness |
| | #5 Isoprene | Discolouration of fingers | Fatigue |
| | | or toes | Itchy skin |
| | | | Yellow skin and eyes |
| 8 | #3 1,3-Butadiene | Shortness of breath | Smokers: |
| | #6 Isobutyronitrile | | Shortness of breath |
| | #7 3-Methylpyridine | | |
| | #8 Pentanoic acid | | |
| | #9 Cresol | | |
| | #11 n-Decane | | |

### Verification of Decision Tree

Table 9 illustrates a table comparing the results obtained using the decision tree generated against reference RT-PCR assay test results, as well as the positive and negative percent agreement of the two tests.

TABLE 9
Comparison of Results from Decision Tree against Reference RT-PCR Results

| | Reference RT-PCR Results | | | | | | | | |
|---|---|---|---|---|---|---|---|---|---|
| Result based on | First Clinical Site | | | Second Clinical Site | | | Total | | |
| Decision Tree | Pos | Neg | Tot | Pos | Neg | Tot | Pos | Neg | Tot |
| Positive | 14 | 6 | 20 | 85 | 5 | 90 | 99 | 11 | 110 |
| Negative | 3 | 153 | 156 | 14 | 200 | 214 | 17 | 353 | 370 |
| Total | 17 | 159 | 176 | 99 | 205 | 304 | 116 | 364 | 480 |

| Positive Percent | 82.4% (95% CI: 63.7%-100.0%) | 85.9% (95% CI: 79.0%-92.8%) | 85.3% (95% CI: 78.9%-91.8%) |
|---|---|---|---|
| Agreement | | | |
| Negative Percent | 96.2% (95% CI: 93.3%-99.2%) | 97.6% (95% CI: 95.4%-99.7%) | 97.0% (95% CI: 95.2%-98.7%) |
| Agreement | | | |
| Positive | 70.0% (95% CI: 49.4%-90.6%) | 94.4% (95% CI: 89.7%-99.2%) | 90.0% (95% CI: 84.4%-95.6%) |
| Predictive Value | | | |
| Negative | 98.1% (95% CI: 95.9%-100.0%) | 93.5% (95% CI: 90.1%-96.8%) | 95.4% (95% CI: 93.3%-97.5%) |
| Predictive Value | | | |

Legend: Pos = Positive; Neg = Negative; Tot = Total
Positive Percent Agreement = True Positives/(True Positives + False Negatives)
Negative Percent Agreement = True Negatives/(True Negatives + False Positives)
Positive Predictive Value = True Positives/(True Positives + False Positives)
Negative Predictive Value = True Negatives/(True Negatives + False Negatives)

The reference RT-PCR identified 116 subjects as COVID-19 positive. On the other hand, the decision tree generated identified 110 subjects to be COVID-19 positive, of which 99 were true positives as confirmed with the reference RT-PCR test. The subjects who were identified to be true COVID-19 positive using the RT-PCR test were categorized and compared to determine a positive percent agreement (PPA) for each category.

Table 10 illustrates a table categorizing subjects identified to be COVID-19 positive by age, as well as a PPA per age bracket. In the First Clinical Site, the clinical trial was conducted on subjects who were either 21 years old or greater, while in the Second Clinical Site, the minimum age of a subject was 18 years old. The results obtained showed that only 3 subjects belonged to the high-risk group of 60 years old and above, while the rest belonged to the age bracket of 6 to 59 years old.

TABLE 10
Positive Results Categorized by Age

| | First Clinical Site | | | Second Clinical Site | | | Total | | |
|---|---|---|---|---|---|---|---|---|---|
| Age | RT- | | PPA | RT- | | PPA | RT- | | PPA |
| (years old) | PCR | DT | (%) | PCR | DT | (%) | PCR | DT | (%) |
| ≤5 | 0 | 0 | NA | 0 | 0 | NA | 0 | 0 | NA |
| 6 to 21 | 1 | 0 | 0 | 9 | 8 | 88.9 | 10 | 8 | 80.0 |
| 22 to 59 | 16 | 14 | 87.5 | 87 | 74 | 85.1 | 103 | 88 | 85.4 |
| ≥60 | 0 | 0 | NA | 3 | 3 | 100 | 3 | 3 | 100 |

Legend: DT = Decision Tree; PPA = Positive Percent Agreement

Table 11 illustrates a table categorizing subjects identified to be COVID-19 positive by smoking history, as well as a PPA per category. A positive percentage agreement (PPA) of 86.3%, 75.0% and 100% were achieved for COVID-19 positive subjects who were non-smokers, current smokers, and ex-smokers, respectively. It was noted that although 6 out of the 11 of the VOCs were found generally in smokers (see Table 7 and Table 8), the PPA of non-smokers were found to be higher than that of the current smokers, indicating that there is no potential interference from smoking activity.

TABLE 11
Positive Results Categorised by Smoking History

| | First Clinical Site | | | Second Clinical Site | | | Total | | |
|---|---|---|---|---|---|---|---|---|---|
| | RT- | | PPA | RT- | | PPA | RT- | | PPA |
| Smoking History | PCR | DT | (%) | PCR | DT | (%) | PCR | DT | (%) |
| Non-smoker | 11 | 9 | 81.8 | 91 | 79 | 86.8 | 102 | 88 | 86.3 |
| Current smoker | 4 | 3 | 75.0 | 8 | 6 | 75.0 | 12 | 9 | 75.0 |
| Ex-smoker | 2 | 2 | 100 | 0 | 0 | NA | 2 | 2 | 100 |

Legend: DT = Decision Tree; PPA = Positive Percent Agreement

Table 12 illustrates a table categorizing the symptomatic subjects based on the number of days since the onset of symptoms, as well as a PPA per category. In the 99 COVID-19 positive subjects in the Second Clinical Site, only 81 subjects presented with symptoms when they visited the clinic. The other 18 COVID-19 positive subjects were asymptomatic when they were tested and underwent the medical examination by doctors at the Second Clinical Site. In the First Clinical Site, 15 COVID-19 positive subjects were symptomatic when they visited the clinic. The clinical information of the other 2 COVID-19 positive subjects were unavailable. In the 96 symptomatic cases, only 2 subjects were found to have the virus for more than 8 days before the onset of symptom while the rest of the subjects displayed COVID-19 symptoms within 8 days of having the virus.

TABLE 12
Positive Results Categorised by Number
of Days Since Onset of Symptoms

| Days Since | First Clinical Site | | | Second Clinical Site | | | Total | | |
|---|---|---|---|---|---|---|---|---|---|
| Symptom | RT- | | PPA | RT- | | PPA | RT- | | PPA |
| Onset | PCR | DT | (%) | PCR | DT | (%) | PCR | DT | (%) |
| <8 days | 14 | 12 | 85.7 | 80 | 69 | 86.3 | 94 | 81 | 86.2 |
| >8 days | 1 | 1 | 100 | 1 | 0 | 0 | 2 | 1 | 50.0 |

Legend: DT = Decision Tree; PPA = Positive Percent Agreement; NA = Not Applicable.

Table 13 illustrates a table comparing the asymptomatic COVID-19 positive subjects identified with the two tests. Among the 127 asymptomatic subjects in the Second Clinical Site who were tested for COVID-19, 18 subjects were found to be positive by RT-PCR test. Among the 18 COVID-19 positive subjects, 16 were correctly detected as COVID-19 positive using the decision tree generated, giving a high positive percent agreement of 89% (Table 13). Similarly, in the 109 COVID-19 negative subjects, all the subjects were correctly detected as COVID-19 negative (100% positive percent agreement). These results showed that the decision tree generated may be used to accurately detect if an asymptomatic subject has COVID-19 or not.

TABLE 13
Asymptomatic COVID-19 Positive Patients

| | Reference RT-PCR Results | | |
|---|---|---|---|
| | Second Clinical Site | | |
| Decision Tree Results | Positive | Negative | Total |
| Positive | 16 | 0 | 16 |
| Negative | 2 | 109 | 111 |
| Total | 18 | 109 | 127 |

| | |
|---|---|
| Positive Percent Agreement | 88.9% (95% CI: 73.9%-100%) |
| Negative Percent Agreement | 100% |

Positive Percent Agreement = True Positives/(True Positives + False Negatives)
Negative Percent Agreement = True Negatives/(True Negatives + False Positives)

A discrepancy analysis was carried out on subjects who were wrongly detected by the decision tree generated, which includes false positives and false negatives. The results showed that there was no commonality or trend within this sub-group population. This sub-group of subjects had a large age range, ranging from 21 years old to 77 years old, were from different ethnic group and different countries. This sub-group also consisted of non-smokers, current smokers, and ex-smokers, indicating that smoking history was not a cause of the discrepancy, which corresponded with the analysis above relating to smoking history (see Table 11). Each subject within this sub-group also presented different symptoms and illness when they visited the clinic. Clinical information available for the Second Clinical Site showed that none of these subjects recruited at the Second Clinical Site had any other respiratory pathogens and hence, ruling out the possibility of interference from other respiratory pathogens. Two RT-PCT kits (Seegene and Perkin Elmer kits) were also used to detect the level of SARS-CoV-2 virus to determine if viral load had any impact on the rate of false positive or false negative. The cycle threshold (Ct) value which was used to determine if an individual had COVID-19 was measured. In general, the higher the Ct values, the lower the viral load an individual has. Table 14 illustrates a table listing the Ct value of 25 randomly selected COVID-19 positive subjects. As illustrated in Table 14, COVID-19 positive subjects who were falsely identified as COVID-19 negative with the decision tree had differing Ct values, ranging from as low as 21.1 to 36.4 when measuring the N gene of the virus using Perkin Elmer kit. Therefore, it appears that the false negative results obtained with the decision tree is not correlated to the Ct value.

TABLE 14
Cycle Threshold (Ct) Values of 25 Randomly Selected COVID-19 Positive Subjects

| | | Seegene | | | | | |
|---|---|---|---|---|---|---|---|
| | Seegene_N | RdRp-S | Seegene_E | Perkin_Or | Perkin | PCR | Prediction with |
| Subject | gene | gene | gene | f gene | N gene | Result | Decision Tree |
| 1 | 21.65 | 21.75 | 21.73 | 19.43 | 21.14 | positive | negative |
| 2 | 17.96 | 18.17 | 18.11 | 19.5 | 21.4 | positive | positive |
| 3 | 21.03 | 21.73 | 20.9 | 21.75 | 23.08 | positive | positive |
| 4 | 18.32 | 18.62 | 18.61 | 21.68 | 23.24 | positive | negative |
| 5 | 17.39 | 17.56 | 17.6 | 27.28 | 23.69 | positive | negative |
| 6 | 21.83 | 17.04 | 17.31 | 21.11 | 22.17 | positive | positive |
| 7 | 22.17 | 21.81 | 21.48 | 19.04 | 21.19 | positive | positive |
| 8 | 24.84 | 24.09 | 24.34 | 23.28 | 25.02 | positive | positive |
| 9 | 24.97 | 25.86 | 24.55 | 22.8 | 22.95 | positive | negative |
| 10 | 25.21 | 23.42 | 23.3 | 23.89 | 29.58 | positive | negative |
| 11 | 25.38 | 27.35 | 26.85 | 24.11 | 22.93 | positive | positive |
| 12 | 26.71 | 24.24 | 24.79 | 24.37 | 26.29 | positive | positive |
| 13 | 26.79 | 27.35 | 26.76 | 28.96 | 30.96 | positive | positive |
| 14 | 27.01 | 26.01 | 25.28 | 20.73 | 22.2 | positive | negative |
| 15 | 27.13 | 21.5 | 21.11 | 23.1 | 25.19 | positive | positive |
| 16 | 32.15 | 36.87 | 33.19 | 27.1 | 28.79 | positive | positive |
| 17 | 32.43 | 31.89 | 33.12 | 29.74 | 31.75 | positive | positive |
| 18 | 30.59 | 29.57 | 28.82 | 27.49 | 29.74 | positive | negative |
| 19 | 34.11 | 35.38 | 33.69 | 30.6 | 30.25 | positive | positive |
| 20 | 30.27 | 29.81 | 29.24 | 33.33 | 31.62 | positive | negative |
| 21 | 29.95 | 30.16 | 29.87 | 29.65 | 32.26 | positive | positive |
| 22 | 30.18 | 31.99 | 30.8 | 34.31 | 33.49 | positive | positive |
| 23 | 34.12 | 35.74 | 35.59 | 33.58 | 34.56 | positive | positive |
| 24 | 33.21 | 34.62 | 33.77 | 33.5 | 35.25 | positive | negative |
| 25 | 34.3 | 35.53 | 34.47 | 36.26 | 36.38 | positive | negative |

Table 15 illustrates a table showing the mean and standard deviation of the false negative and true positive groups. As illustrated in Table 15, the mean and standard deviation of the false negative and true positive groups were very similar across all the genes tested, thus indicating that there is no correlation between the Ct value and false negative.

TABLE 15
Cycle threshold (Ct) Values

| | | Ct values of different genes | | | | |
|---|---|---|---|---|---|---|
| | | | Seegene | | | |
| | | Seegene_N | RdRp-S | Seegene_E | Perkin_Orf | Perkin |
| Tests | Statistics | gene | gene | gene | gene | N gene |
| COVID-Positive | Mean | 25.7 | 25.6 | 25.1 | 24.3 | 25.3 |
| Classification | Standard | 5.6 | 6.0 | 5.9 | 5.0 | 5.0 |
| with Decision Tree | Deviation | | | | | |
| COVID-19 Negative | Mean | 25.8 | 25.8 | 25.3 | 25.3 | 26.5 |
| Classification | Standard | 5.5 | 5.7 | 5.4 | 6.1 | 5.8 |
| with Decision Tree | Deviation | | | | | |

Thus, with the data that was available, no trend could be found for this group of subjects that resulted in the discrepancy between results obtained from the decision tree generated and the RT-PCR test.

A cross-reactivity study was carried out to identify if the decision tree generated also identified the presence of viral infections other than COVID-19. Some samples obtained from subjects were tested for a panel of high prevalence respiratory pathogens that may potentially cross-react with the method in the present disclosure. The results are presented below in Table 16. The performance of the method disclosed in the present disclosure on these samples were all negative.

TABLE 16
Cross-Reactivity Performance

| | No. of | COVID-19 RT- | | COVID-19 RT- | |
|---|---|---|---|---|---|
| | Patients | PCR Negative | | PCR Positive | |
| | with | | DT | | DT |
| Potential Cross-Reactant | Pathogens | DT Pos | Neg | DT Pos | Neg |
| Influenza A-H3 (Flu A-H3) | 1 | 0 | 1 | 0 | 0 |
| Respiratory syncytial virus A (RSV A) | 1 | 0 | 1 | 0 | 0 |
| Enterovirus (HEV) | 3 | 0 | 3 | 0 | 0 |
| Parainfluenza virus 1 (PIV 1) | 1 | 0 | 1 | 0 | 0 |
| Parainfluenza virus 4 (PIV 4) | 1 | 0 | 1 | 0 | 0 |
| Coronavirus NL63 (NL63) | 1 | 0 | 1 | 0 | 0 |
| Coronavirus OC43 (OC43) | 2 | 0 | 2 | 0 | 0 |
| Human rhinovirus (HRV) | 7 | 0 | 7 | 0 | 0 |
| Haemophilus influenzae (HI) | 1 | 0 | 0 | 1 | 0 |
| Streptococcus pneumoniae (SP) | 2 | 0 | 0 | 2 | 0 |

Legend:
DT = Decision Tree;
Pos = Positive;
Neg = Negative.

An interference study was carried out to determine if the decision tree generated also identified subjects with lung cancer and tuberculosis. Table 17 illustrates results obtained from samples of subjects with lung cancer and tuberculosis but without COVID-19. Although the data structure of breath samples of these patients differed from the samples obtained with the method presently disclosed, the results obtained showed that there was no interference from other diseases like lung cancer and tuberculosis.

TABLE 17
Classification of Samples from subjects with lung cancer and tuberculosis

| | | COVID-19 | COVID-19 | Negative |
|---|---|---|---|---|
| | No of | Positive | Negative | Percent |
| Disease | Samples | Classification | Classification | Agreement |
| Lung Cancer | 29 | 0 | 29 | 100 |
| Tuberculosis | 11 | 0 | 11 | 100 |

### Repeatability of Method

The repeatability of the method disclosed, including sample collection, biomarker analysis, and classification with the decision tree was determined. The repeatability was determined by analysing multiple samples from subjects who were confirmed to be true COVID-19 positive or true COVID-19 negative. Each breath was analysed separately, and the concentrations detected were applied through the decision tree to arrive at a classification of COVID-19 positive or COVID-19 negative. For subjects who were confirmed to be true COVID-19 positive, an average coefficient of variation was carried out for each biomarker.

Table 18 illustrates a table showing the results of a repeatability test carried out on subjects who were identified to be true COVID-19 negative. According to the data obtained, the method disclosed was able to correctly classify most breath samples as true negative COVID-19 samples. Only 1 subject had breath samples that were falsely classified as COVID-19 positive. The negative percent agreement was calculated to be 97.5% which is in well agreement with that obtained previously (see Table 9). The standard deviation for the mean was also very small (between 0.00-0.3) which indicated a high repeatability of the method disclosed.

TABLE 18
Analysis of Breath Samples

| | PTR-MS | | | | Mean ± |
|---|---|---|---|---|---|
| | TOF | No of | DT | DT | S.D. of DT |
| S/N | Model | Breaths | Negative | Positive | Negative |
| Subject 1 | 1000 | 50 | 45 | 5 | 0.90 ± 0.30 |
| Subject 2 | 1000 | 50 | 50 | 0 | 1.00 ± 0.00 |
| Subject 3 | 1000X | 50 | 50 | 0 | 1.00 ± 0.00 |
| Subject 4 | 1000X | 50 | 50 | 0 | 1.00 ± 0.00 |

| | | | | |
|---|---|---|---|---|
| Total | 200 | 195 | 5 | 0.98 ± 0.16 |

| | |
|---|---|
| Negative Percent Agreement | 97.5% |

Negative Percent Agreement = True Negatives/(True Negatives ± False Positives);
DT = Decision Tree

Table 19 illustrates a table showing the results of a repeatability test carried out on subjects who were identified to be true COVID-19 positive. A total of 20 subjects were analysed, with 3 samples from each subject analysed. In order to show the extent of variability in relation to the mean of the population, the coefficient of variations (CV), a standardized measure of dispersion of a probability distribution, were calculated for each biomarker in each subject based on the ratio of the standard deviation (o) to the mean (u) using equation (vi) below:

$$CV = \frac{σ}{μ} \qquad ( vi )$$

An average of the CV values obtained for each biomarker was then obtained.

TABLE 19
Average coefficient of variations (CV) of 20 COVID-19
positive patients for each biomarker

| m/z | Biomarker | CAS Number | Average CV (%) |
|---|---|---|---|
| 38.04 | Water (Cluster) | 7732-18-5 | 2.63 |
| 51.04 | Methanol (Water Cluster) | 67-56-1 | 6.53 |
| 55.05 | 1,3-Butadiene | 106-99-0 | 5.44 |
| 59.05 | Acetone | 67-64-1 | 2.99 |
| 69.08 | Isoprene | 78-79-5 | 4.58 |
| 70.07 | Isobutyronitrile | 78-82-0 | 5.27 |
| 94.07 | 3-Methylpyridine | 108-99-6 | 5.85 |
| 103.08 | Pentanoic Acid | 109-52-4 | 6.21 |
| 109.07 | m-Cresol or p-Cresol | 108-39-4 | 5.49 |
| | | 106-44-5 | |
| 121.96 | N,N-Dimethylaniline | 121-69-7 | 6.80 |
| 143.18 | n-Decane | 124-18-5 | 6.01 |

As shown in Table 19 above, the average CV for each biomarker was all below 10%, indicating that the variability of each breath was low. Hence, the results showed that the method disclosed in the present application has high repeatability for COVID-19 positive patients.

### Ensemble Machine Learning Model

An ensemble machine learning model was built on exhaled VOCs for COVID-19 diagnosis. The machine learning comprises constructing a dataset, pre-processing data and thereby building a machine learning model. The overall result of the model built was subsequently validated to determine the robustness of the implemented method.

### Dataset Construction

The dataset contains 1496 samples collected at three venues. The samples were analyzed using a PTR-TOF-MS (Ionicon, PTR-TOF 4000). During data collection, each subject was asked to perform at least one complete respiratory cycle on the PTR-MS machine, and be examined by COVID-19 PCR test. The data of each sample contains: collection date, full PTR-MS spectra against time, PCR test result (positive or negative), other recorded data of PTR-MS operation. The statistics of sample subjected to the PCR test are shown in Table 20.

TABLE 20
Results of the PCR test

| PCR Result | Sample Number |
|---|---|
| Positive | 550 |
| Negative | 946 |

Subsequently, the dataset was randomly split by train/test set with the ratio 80%/20% as indicated in Table 21. As mentioned above, other suitable ratios between the train and test sets may be used.

TABLE 21
Random splitting of train and test sets

| PCR Result | # in Train Set | # in Test Set |
|---|---|---|
| Positive | 440 | 110 |
| Negative | 758 | 188 |

Peaks were extracted from the spectra by peak detection algorithms in IONICON viewer software. It is noted that the intensity of extracted peaks was measured in ion counts (intensity values). Thus, further transformations were performed on the intensity value. The transformations utilize PTR-transmission information and recorded reaction rate to convert the intensity into concentration (Unit: ppb). Overall, a total 2344 tracing peaks with their corresponding concentrations were extracted for each data sample.

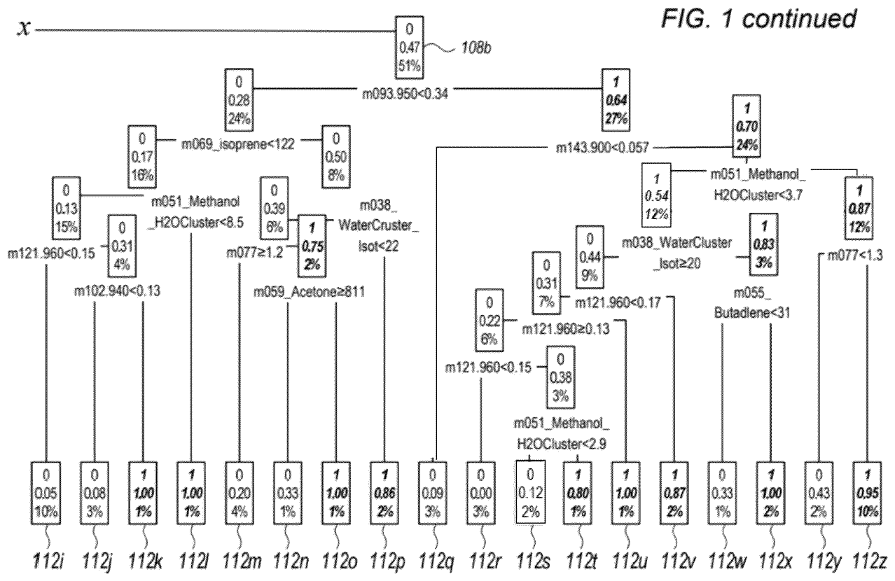

The spectra of respiratory cycles were segmented into three phases, namely, background, exhaling and unclassified as shown in FIG. 2. Such segmentation was conducted by performing median filtering, normalization, and quantization on the several tracing VOCs throughout the respiratory cycle. The selected tracing VOCs were Acetone (m/z 59.05) and Isoprene (m/z 69.07). A machine learning model was developed to diagnose VOCs-related disease by PTR-MS breath data. To address the possible over-fitting, non-normalization and label imbalance problem, the utilized machine learning model is selected as an ensemble model of decision trees i.e. Extreme Gradient Boosting (or XGBoosting). This model is an ensemble learning method for classification that operates by constructing a multitude of decision trees at training time. For classification tasks, the output of the XGBoosting is the sum of all tree predictions. It is to be understood other suitable model including random forest model may be used.

After training process, the learned machine learning model performs classification on the testing set. In Table 22, the performance of the output model for the purpose of COVID-19 diagnosis by multiple clinical measurements is presented. It can be seen from Table 22 that the trained model displays high sensitivity, specificity and predictive values.

TABLE 22
Performance of the output model for COVID-19 diagnosis

| Name | Value | Lower CI | Upper CI |
|---|---|---|---|
| Sensitivity | 85.5% | 77.7% | 90.8% |
| Specificity | 96.3% | 92.5% | 98.2% |
| Positive Predictive Value | 93.1% | 86.4% | 96.6% |
| Negative Predictive Value | 91.9% | 87.2% | 94.9% |

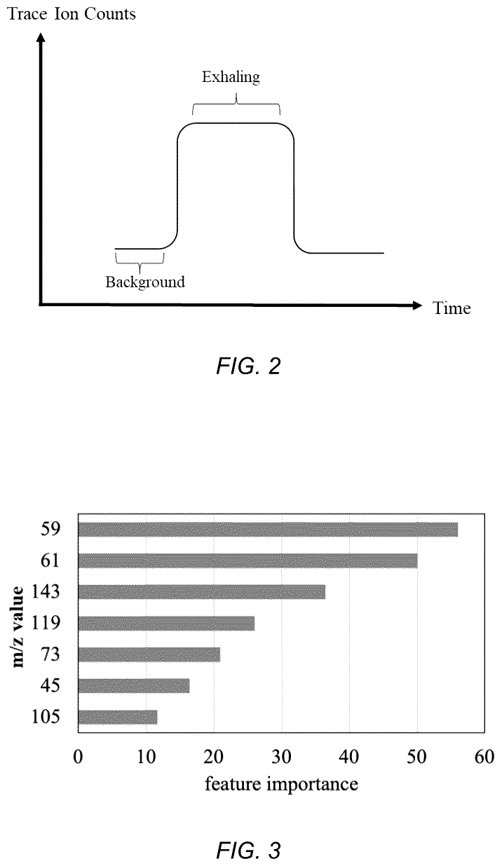

Importance factors for the VOCs in the output model were calculated by averaging the factors across all decision trees in the model. Specifically, the importance of a m/z value (feature) is calculated by summing the Gains of the feature in all trees of XGBoosting. Gain is the improvement in accuracy brought by a feature to the branches during the training process. The selected features with significant importance and the values of their molecular weight are displayed in FIG. 3. The information gain of one node was calculated using equation (vii) as follows:

$$Gain = \frac{1}{2} [ \frac{{G}_{L}^{2}}{{H}_{L} + λ} + \frac{{G}_{R}^{2}}{{H}_{R} + λ} - \frac{{( {G}_{L} + {G}_{R} )}^{2}}{{H}_{L} + {H}_{R} + λ} ] \qquad ( vii )$$

Where G_{L} and G_{R} are the summed gradient gain of left child and right child, respectively. H_{L} and H_{R} denote the summed second-order gradient gain. They were calculated by summing all gains of the selected samples of a child node. λ is a tunable parameter, which is set as the default value being 1. The values of gains may depend on the training dataset and model setting. To interpret the XGBoosting model of biomarkers or VOCs, the most important concentration thresholds were extracted for all selected biomarkers or VOCs. The threshold of a biomarker or VOC was determined by the following procedure: extracting all decision nodes with thresholds of the biomarker or VOC; extracting the information gain of each node and selecting the threshold with the largest gain among all. It is to be understood that the method of obtaining the concentration threshold is not limited to the above steps. The above steps were used for their simplicity and interpretability. Therefore, the thresholds obtained as will be described below may be subject to change for different conditions. The changes may arise from the air environment where the data are collected,

Additionally, the identification of the biomarkers represented by these seven biomarkers or VOCs is shown in Table 23. As can be seen from Table 23, the samples that were tested positive show increase in the concentration level for some of the biomarkers compared to the samples being tested negative (Acetone having an increase of about 1.4 times, 1-Propanol with an increase of about 1.2 times, Nonanal with an increase of about 2 times, 3-(Ethylthio)propanal with an increase of about 2.5 times, and Acetaldehyde with an increase of about 4 times). Further, as can be seen from Table 23, the concentration of some biomarkers in the positive tested sample is lower than that in the negative tested sample (Butyraldehyde with a decrease of about 0.5 times and 3-Hydroxybutyric Acid with a decrease of about 0.5 times). Based on the model above, the concentration threshold (in ppb) for the biomarkers to be categorized as COVID positive is as follow: Acetone with a concentration greater than 870 ppb, 1-Propanol with a concentration greater than 35 ppb, Nonanal with a concentration greater than 0.27 ppb, 3-(Ethylthio)propanal with a concentration greater than 0.5 ppb, Butyraldehyde with a concentration greater than 0.05 ppb, Acetaldehyde with a concentration lower than 1 ppb and 3-Hydroxybutyric Acid with a concentration higher than 0.1 ppb. As can be appreciated, when a sample is identified to have Acetone with a concentration greater than 870 ppb and 1-Propanol with a concentration greater than 35 ppb, it may be sufficient to presume that the sample is COVID positive. Therefore, advantageously, the method disclosed herein may not require the identification and/or quantification of all seven (7) biomarkers as shown in Table 23 below. This may similarly apply to the first list, second list, third list and fourth list of biomarkers described above.

TABLE 23
Fifth List of Biomarkers Identified

| | | | (+) Samples | (−) Samples |
|---|---|---|---|---|
| S/N | m/z | Biomarker | (ppb) | (ppb) |
| 1 | 59.05 | Acetone | 320~910 | 260~650 |
| 2 | 61.10 | 1-Propanol | 16~38 | 15~33 |
| 3 | 143.24 | Nonanal | 0~0.6 | 0~0.3 |
| 4 | 119.19 | 3-(Ethylthio)propanal | 0~0.5 | 0~0.2 |
| 5 | 73.06 | Butyraldehyde | 0~0.07 | 0~0.14 |
| 6 | 45.05 | Acetaldehyde | 0~4 | 0~1 |
| 7 | 105.11 | 3-Hydroxybutyric Acid | 0~0.1 | 0~0.17 |

It should be appreciated that the above-described methods may be varied in many ways, including omitting or adding steps, changing the order of steps and the type of devices used. It should be appreciated that different features may be combined in different ways. In particular, not all the features shown above in a particular embodiment are necessary in every embodiment of the disclosure. Further combinations of the above features are also considered to be within the scope of some embodiments of the disclosure.

It will be appreciated by persons skilled in the art that the present invention is not limited to what has been particularly shown and described hereinabove. Rather the scope of the present invention is defined only by the claims, which follow.

1. A method of detecting the presence of COVID-19 in a subject, the method comprising the steps of:
detecting and/or measuring concentrations of the at least two biomarkers in a breath sample of the subject; and
comparing the concentrations to that in COVID-19 negative individuals;
wherein the subject is COVID-19 positive if the concentrations of each of the at least two biomarkers are higher than concentrations in COVID-19 negative individuals.

2. The method of claim 1, wherein the at least two biomarkers comprise acetone and 1-propanol, and wherein the subject is COVID-19 positive if the concentrations of acetone and 1-propanol are about 1.4 times higher and 1.2 times higher when compared to COVID-19 negative individuals respectively.

3. The method of claim 2, wherein the at least two biomarkers are detected/quantified by mass spectrometry wherein a m/z value of acetone is 59.05±1 and a m/z value of 1-Propanol is 61.10±1.

4. The method of claim 2, wherein the at least two biomarkers further comprise nonanal, 3-(ethylthio)propanal and acetaldehyde.

5. The method according to claim 1, wherein the at least two biomarkers comprise butanol and isoprene, and wherein the subject is COVID-19 positive if the concentrations of butanol and isoprene are about 3 times higher and about 1.8 times higher than in COVID-19 negative individuals respectively.

6. (canceled)

7. The method of claim 5, wherein the at least two biomarkers are detected and/or quantified by mass spectrometry, wherein a m/z value of butanol is 75.12±1 and a m/z value of isoprene is 69.07±1.

8. The method of claim 5, wherein the at least two biomarkers further comprise acetone, cycloheptene, and sesquiterpene.

9. The method according to claim 1, wherein the at least two biomarkers comprise 1,3-butadiene and acetone, wherein the subject is COVID-19 positive if the concentrations of 1,3-butadiene and acetone are about 1.5 times higher about 1.7 higher than in COVID-19 negative individuals respectively.

10. (canceled)

11. The method of claim 9, wherein the at least two biomarkers are detected/quantified by mass spectrometry, wherein a m/z value of 1,3-butadiene is 55.05±1 and a m/z value of acetone is 59.05±1.

12. The method of claim 9, wherein at least two biomarkers further comprises isoprene propanoic acid, aniline, 1-Octene, decene, dodecane, β-damascenone, sesquiterpene, and tetradecanoic acid.

13. A method of detecting the presence of COVID-19 in a subject, the method comprising the steps of:
detecting and/or measuring at least two biomarkers comprising hydrogen peroxide and water in a breath sample of the subject; and
comparing the concentrations to that in COVID-19 negative individuals;
wherein the concentrations of hydrogen peroxide and water is lower and higher than in COVID-19 negative individuals respectively.

14. The method of claim 13, wherein the at least two biomarkers further comprises 1,3-butadiene, acetone, trimethylamine, isoprene, carbon disulfide, 3-methylpyridine, benzoic acid, and tetradecanoic acid.

15. (canceled)

16. (canceled)

17. The method of claim 1, wherein said biomarkers are detected and measured by proton transfer reaction time-of-flight mass spectrometry (PTR-TOF-MS).

18. (canceled)

19. (canceled)

20. (canceled)

21. (canceled)

22. A method of detecting the presence of a respiratory disease in a subject, the method comprising the steps of:
(a) collecting a breath sample from a subject;
(b) detecting in said breath sample two or more biomarkers and measuring a concentration of the two or more biomarkers, wherein said biomarkers are selected from the group consisting of water, methanol, 1,3-butadiene, acetone, isoprene, isobutyronitrile, 3-methylpyridine, pentanoic acid, m-cresol or p-cresol, N,N-dimethylaniline, and n-decane; and
(c) determining the presence of a respiratory disease.

23. The method according to claim 1, wherein the at least two biomarkers comprise n-decane and isobutyronitrile.

24. The method of claim 2, further comprising the steps of:
detecting or measuring concentrations of butyraldehyde and 3-hydroxybutyric acid; and
comparing the concentrations to that in COVID-19 negative individuals;
wherein the subject is COVID-19 positive if concentrations of butyraldehyde and/or 3-hydroxybutyric acid are lower than concentrations in COVID-19 negative individuals.

25. The method of claim 12, further comprising the steps of:
detecting and/or measuring additional biomarkers, wherein the additional biomarkers are selected from a group consisting of methacrolein, butanal, furfural and 6-methyl-5-hepten-2-one, and
comparing the concentrations to that in COVID-19 negative individuals;
wherein the subject is COVID-19 positive if concentrations of the additional biomarkers are lower than in COVID-19 negative individuals.

26. The method of claim 23, wherein the at least two biomarkers further comprise isoprene.
